(19) United States
(12) Patent Application Publication
(10) Pub. No.: US 2026/0062681 A1
(43) Pub. Date: Mar. 5, 2026

(54) RECOMBINANT ADENO-ASSOCIATED VIRUS PRODUCTS AND METHODS FOR TREATING LIMB GIRDLE MUSCULAR DYSTROPHY 2A

(71) Applicant: RESEARCH INSTITUTE AT NATIONWIDE CHILDREN'S HOSPITAL, Columbus, OH (US)

(72) Inventor: Zarife Sahenk, Columbus, OH (US)

(73) Assignee: RESEARCH INSTITUTE AT NATIONWIDE CHILDREN'S HOSPITAL, Columbus, OH (US)

(21) Appl. No.: 19/261,876

(22) Filed: Jul. 7, 2025

Related U.S. Application Data
(63) Continuation of application No. 17/255,488, now Pat. No. 12,391,928.
(60) Provisional application No. 62/691,934, filed on Jun. 29, 2018.
(60) Provisional application No. 62/865,081, filed on Jun. 21, 2019.

(51) Int. Cl. C12N 7/00; A61K 48/00; A61P 21/00; C12N 9/64; C12N 15/86

(52) U.S. Cl. CPC C12N 7/00; A61K 48/0058; A61P 21/00; C12N 9/6472; C12N 15/86; C12N 2750/14131; C12N 2750/14143

(57) ABSTRACT

Products and methods for treating limb girdle muscular dystrophy 2A are provided. In the methods, recombinant adeno-associated viruses deliver DNA encoding a protein with calpain 3 activity.

This application is a continuation of U.S. patent application Ser. No. 17/255,488, filed Dec. 23, 2020, now U.S. Pat. No. 12,391,928, which is a U.S. 371 National Stage Application of PCT International Application No. PCT/US19/39893, filed Jun. 28, 2019, which claims priority to U.S. Provisional Patent Application No. 62/691,934, filed Jun. 29, 2018 and U.S. Provisional Patent Application No. 62/865,081, filed Jun. 21, 2019, the contents of each of which are incorporated herein by reference in their entirety.

### FIELD

Provided herein are products and methods for treating limb girdle muscular dystrophy 2A. In the methods, recombinant adeno-associated viruses deliver DNA encoding a protein with calpain3 (CAPN3) activity.

### INCORPORATION BY REFERENCE OF THE SEQUENCE LISTING

This application contains, as a separate part of disclosure, a Sequence Listing in XML format (filename: 140056-0836_SL.xml; 28,677 bytes in size, created on Nov. 18, 2025) which is incorporated by reference herein in its entirety.

### BACKGROUND

Muscular dystrophies (MDs) are a group of genetic diseases. The group is characterized by progressive weakness and degeneration of the skeletal muscles that control movement. Some forms of MD develop in infancy or childhood, while others may not appear until middle age or later. The disorders differ in terms of the distribution and extent of muscle weakness (some forms of MD also affect cardiac muscle), the age of onset, the rate of progression, and the pattern of inheritance.

One group of MDs is the limb girdle group (LGMD) of MDs. LGMDs are rare conditions and they present differently in different people with respect to age of onset, areas of muscle weakness, heart and respiratory involvement, rate of progression and severity. LGMDs can begin in childhood, adolescence, young adulthood or even later. Both genders are affected equally. LGMDs cause weakness in the shoulder and pelvic girdle, with nearby muscles in the upper legs and arms sometimes also weakening with time. Weakness of the legs often appears before that of the arms. Facial muscles are usually unaffected. As the condition progresses, people can have problems with walking and may need to use a wheelchair over time. The involvement of shoulder and arm muscles can lead to difficulty in raising arms over head and in lifting objects. In some types of LGMD, the heart and breathing muscles may be involved.

There are at least nineteen forms of LGMD, and the forms are classified by their associated genetic defects.

| Type | Pattern of Inheritance | Gene or Chromosome |
|---|---|---|
| LGMD1A | Autosomal dominant | Myotilin gene |
| LGMD1B | Autosomal dominant | Lamin A/C gene |
| LGMD1C | Autosomal dominant | Caveolin gene |
| LGMD1D | Autosomal dominant | Chromosome 7 |
| LGMD1E | Autosomal dominant | Desmin gene |
| LGMD1F | Autosomal dominant | Chromosome 7 |
| LGMD1G | Autosomal dominant | Chromosome 4 |
| LGMD2A | Autosomal recessive | Calpain-3 gene |
| LGMD2B | Autosomal recessive | Dysferlin gene |
| LGMD2C | Autosomal recessive | Gamma-sarcoglycan gene |
| LGMD2D | Autosomal recessive | Alpha-sarcoglycan gene |
| LGMD2E | Autosomal recessive | Beta-sarcoglycan gene |
| LGMD2F | Autosomal recessive | Delta-sarcoglycan gene |
| LGMD2G | Autosomal recessive | Telethonin gene |
| LGMD2H | Autosomal recessive | TRIM32 |
| LGMD2I | Autosomal recessive | FKRP gene |
| LGMD2J | Autosomal recessive | Titin gene |
| LGMD2K | Autosomal recessive | POMT1 gene |
| LGMD2L | Autosomal recessive | Fukutin gene |

Specialized tests for LGMD are now available through a national scheme for diagnosis, the National Commissioning Group (NCG).

Mutations in calpain3 gene (CAPN3) lead to one of the most common limb-girdle muscular dystrophies worldwide, LGMD2A. At present, there is no treatment for this inherited disease. Previous studies have demonstrated the potential for CAPN3 gene transfer to correct the pathological signs in CAPN3-deficient mice. However expression of CAPN3 driven by desmin promoter resulted in cardiotoxicity [Bartoli et al., Mol. Ther., 13: 250-259 (2006)]. In follow up studies, skeletal muscle expression of the gene was studied [Roudaut et al., Circulation, 128: 1094-1104 (2013)].

Adeno-associated virus (AAV) is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of AAV serotypes are known. For example, the complete genome of AAV-1 is provided in GenBank Accession No. NC_002077; the complete genome of AAV-2 is provided in GenBank Accession No. NC 001401 and Srivastava et al., J. Virol., 45: 555-564 {1983); the complete genome of AAV-3 is.provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in see U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

There remains a need in the art for treatments for LGMD2A.

### SUMMARY

Methods and products for delivering DNA encoding a protein with calpain3 (CAPN3) activity are provided herein. Such methods and products can be used to treat various diseases, for example, LGMD2A.

Recombinant adeno-associated viruses (rAAVs) are provided encoding a protein with calpain 3 (CAPN3) activity. The recombinant adeno-associated viruses comprise a polynucleotide that comprises a nucleotide sequence encoding the protein with CAPN3 activity. The nucleotide sequence encoding the protein with CAPN3 activity, for example, is at least 90% identical to SEQ ID NO: 2 or comprises the sequence of SEQ ID NO: 2.

For example, the provided rAAV comprise a polynucleotide which comprises a first AAV inverted terminal repeat (ITR), a promoter, a nucleotide sequence encoding a protein with calpain 3 (CAPN3) activity and a second AAV ITR. The nucleotide sequence encoding the protein with CAPN3 activity, for example, is at least 90% identical to SEQ ID NO: 2, or at least 91% identical to SEQ ID NO: 2, at least 92% identical to SEQ ID NO: 2, at least 93% identical to SEQ ID NO: 2, at least 94% identical to SEQ ID NO: 2, at least 95% identical to SEQ ID NO: 2, at least 96% identical to SEQ ID NO: 2, at least 97% identical to SEQ ID NO: 2, at least 98% identical to SEQ ID NO: 2, or at least 99% identical to SEQ ID NO: 2. The rAAV comprises a nucleotide sequence encoding a protein with CAPN3 activity comprises the sequence of SEQ ID NO: 2.

In addition, the provided rAAV comprises a nucleotide sequence encoding a protein with CAPN3 activity that comprises an amino acid sequence that is at least 90% identical to SEQ ID NO:7, at least 91% identical to SEQ ID NO:7, at least 92% identical to SEQ ID NO:7, at least 93% identical to SEQ ID NO:7, at least 94% identical to SEQ ID NO: 7, at least 95% identical to SEQ ID NO:7, at least 96% identical to SEQ ID NO:7, at least 97% identical to SEQ ID NO:7, at least 98% identical to SEQ ID NO:7, or at least 99% identical to SEQ ID NO: 7. The rAAV comprises a nucleotide sequence encoding a protein with CAPN3 activity comprising the amino acid sequence of SEQ ID NO: 7.

The provided rAAV comprise a polynucleotide sequence which is at least 90% identical to SEQ ID NO: 1, at least 91% identical to SEQ ID NO: 1, at least 92% identical to SEQ ID NO: 1, at least 93% identical to SEQ ID NO: 1, at least 94% identical to SEQ ID NO: 1, at least 95% identical to SEQ ID NO: 1, at least 96% identical to SEQ ID NO: 1, at least 97% identical to SEQ ID NO: 1, at least 98% identical to SEQ ID NO: 1, or at least 99% identical to SEQ ID NO: 1. The rAAV comprises a polynucleotide sequence of SEQ ID NO: 1.

The nucleotide sequence, in one embodiment, is under the transcription control of a muscle-specific promoter. For example, the muscle-specific promoter comprises one or more of a human skeletal actin gene element, a cardiac actin gene element, a desmin promoter, a skeletal alpha-actin (ASKA) promoter, a troponin I (TNNI2) promoter, a myocyte-specific enhancer binding factor mef binding element, a muscle creatine kinase (MCK) promoter, a truncated MCK (tMCK) promoter, a myosin heavy chain (MHC) promoter, a hybrid a-myosin heavy chain enhancer-/MCK enhancer-promoter (MHCK7) promoter, a C5-12 promoter, a murine creatine kinase enhancer element, a skeletal fast-twitch troponin c gene element, a slow-twitch cardiac troponin c gene element, a slow-twitch troponin i gene element, hypoxia-inducible nuclear factor (HIF)-response element (HRE), a steroid-inducible element, and a glucocorticoid response element (gre). In one embodiment, the muscle-specific promoter is a tMCK promoter, which comprises a sequence of SEQ ID NO: 3.

For example, the rAAV comprises a polynucleotide which comprises, in one embodiment, a first AAV inverted terminal repeat (ITR), a tMCK promoter, the nucleotide sequence encoding the protein with calpain 3 activity, and a second AAV inverted terminal repeat (ITR). The AAV ITR (e.g., the first and/or second AAV ITRs) is, for example, an AAV2 inverted terminal repeat. The capsid proteins of the rAAV comprise, for example, an AAV rh. 74 capsid protein or an AAV9 capsid protein.

The provided rAAV comprises one or more of AAV-1, AAV-2, AAV-3, AAV-4, AAV-5, AAV-6, AAV-7, AAV-8, AAV-9, AAV-10, AAV-11, AAV-12, AAV-13, AAV rh.74 and AAV rh. 10 capsid proteins.

In another embodiment, compositions comprising any of the disclosed rAAV are provided. For example, the compositions are formulated for intramuscular injection or intravenous injection.

Methods of treating limb girdle muscular dystrophy 2A in a subject comprising administering to the subject a therapeutically effective amount of any of the disclosed rAAV or any composition comprising a disclosed rAAV are also provided. In any of the provided methods, the rAAV are administered by intramuscular injection or intravenous injection.

For example, in these methods treatment results in one or more of: (a) an increased muscle fiber diameter, (b) a decreased number of small lobulated muscle fibers, (c) a decreased number of fibers with internal nuclei, (d) a decreased endomysial connective tissue content, (e) correction of muscle atrophy, and (f) a increased muscle force generation. The muscle fiber affected by the treatment comprise one or more of slow twitch oxidative (STO) muscle fiber, fast twitch oxidative (FTO) muscle fiber, and fast twitch glycolytic (FTG) fiber.

In addition, in any of the provided methods, the treatment results in one or more of: (a) at least a 5%, 10%, 15%, 20%, 25%, 30%, or 35%, or 40% decrease of total muscle fiber number per mm² by 4 weeks after administration; (b) at least a 5%, 10%, 15%, 20%, or 25% increase of muscle fiber diameter by 4 weeks after administration; (c) at least a 5%, 10%, 15%, 20%, 25%, 30%, 35%, or 42% decrease of STO muscle fiber number per mm² by 4 weeks after administration; (d) at least a 5%, 10%, 15%, 20%, or 25% increase of STO muscle fiber diameter by 4 weeks after administration; (e) at least a 5%, 10%, 15%, or 20% decrease of FTO muscle fiber number per mm² by 4 weeks after administration; (f) at least a 5%, 10%, 15%, or 20% increase of FTO muscle fiber diameter by 4 weeks after administration; (g) at least a 5%, 10%, 15%, 20%, 25%, 30%, or 35% decrease of FTG muscle fiber number per mm² by 4 weeks after administration; and (h) at least a 5%, 10%, 15%, 20%, or 25% increase of FTG muscle fiber diameter by 4 weeks after administration.

In any of the provided methods, the heart muscle of the subject shows minimum or low calpain 3 protein expressed from any of the provided rAAV, or a composition comprising any of the provided rAAV. The muscle fiber affected by the treatment with the composition comprise one or more of slow twitch oxidative (STO) muscle fiber, fast twitch oxidative (FTO) muscle fiber, and fast twitch glycolytic (FTG) fiber.

Compositions for treating limb girdle muscular dystrophy 2A comprising a therapeutically effective amount of any of the disclosed rAAV or a composition comprising any of the disclosed rAAV are provided. These composition for treating treating limb girdle muscular dystrophy 2A are formulated for administration by intramuscular injection or intravenous injection. In addition, treatment with any of the disclosed compositions limb girdle muscular dystrophy 2A results in one or more of: (a) an increased muscle fiber diameter, (b) a decreased number of small lobulated muscle fibers, (c) a decreased number of fibers with internal nuclei, (d) a decreased endomysial connective tissue content, (e) correction of muscle atrophy, and (f) a increased muscle force generation. The muscle fiber affected by the treatment with the composition comprise one or more of slow twitch oxidative (STO) muscle fiber, fast twitch oxidative (FTO) muscle fiber, and fast twitch glycolytic (FTG) fiber.

In addition, the treatment with any of the disclosed compositions for treating limb girdle muscular dystrophy 2A results in one or more of: (a) at least a 5%, 10%, 15%, 20%, 25%, 30%, or 35%, or 40% decrease of total muscle fiber number per mm² by 4 weeks after administration; (b) at least a 5%, 10%, 15%, 20%, or 25% increase of muscle fiber diameter by 4 weeks after administration; (c) at least a 5%, 10%, 15%, 20%, 25%, 30%, 35%, or 42% decrease of STO muscle fiber number per mm² by 4 weeks after administration; (d) at least a 5%, 10%, 15%, 20%, or 25% increase of STO muscle fiber diameter by 4 weeks after administration; (e) at least a 5%, 10%, 15%, or 20% decrease of FTO muscle fiber number per mm² by 4 weeks after administration; (f) at least a 5%, 10%, 15%, or 20% increase of FTO muscle fiber diameter by 4 weeks after administration; (g) at least a 5%, 10%, 15%, 20%, 25%, 30%, or 35% decrease of FTG muscle fiber number per mm² by 4 weeks after administration; and (h) at least a 5%, 10%, 15%, 20%, or 25% increase of FTG muscle fiber diameter by 4 weeks after administration.

Further, treatment with any of the provided compositions for treatment of limb girdle muscular dystrophy 2A results in the heart muscle of the subject showing minimum or low calpain 3 protein expressed from any of the provided rAAV, or a composition comprising any of the provided rAAV. The heart muscle, after administration with the rAAV, shows no or little toxic effect, e.g., inflammation, necrosis and/or regeneration.

The disclosure also provides for use of a therapeutically effective amount of any of the disclosed rAAV or a composition comprising any of the disclosed rAAV for the preparation of a medicament for the treatment of limb girdle muscular dystrophy 2A. For example, the medicament is formulated for administration by intramuscular injection or intravenous injection.

In any of the uses, treatment with the medicament results in one or more of: (a) an increased muscle fiber diameter, (b) a decreased number of small lobulated muscle fibers, (c) a decreased number of fibers with internal nuclei, (d) a decreased endomysial connective tissue content, (e) correction of muscle atrophy, and (f) a increased muscle force generation. The muscle fiber affected by treatment with the medicament is one or more of slow twitch oxidative (STO) muscle fiber, fast twitch oxidative (FTO) muscle fiber, and fast twitch glycolytic (FTG) fiber.

In addition, in any of the uses of a therapeutically effective amount of any of the disclosed rAAV or a composition provided, treatment with the medicament results in one or more of: (a) at least a 5%, 10%, 15%, 20%, 25%, 30%, or 35%, or 40% decrease of total muscle fiber number per mm² by 4 weeks after administration; (b) at least a 5%, 10%, 15%, 20%, or 25% increase of muscle fiber diameter by 4 weeks after administration; (c) at least a 5%, 10%, 15%, 20%, 25%, 30%, 35%, or 42% decrease of STO muscle fiber number per mm² by 4 weeks after administration; (d) at least a 5%, 10%, 15%, 20%, or 25% increase of STO muscle fiber diameter by 4 weeks after administration; (e) at least a 5%, 10%, 15%, or 20% decrease of FTO muscle fiber number per mm² by 4 weeks after administration; (f) at least a 5%, 10%, 15%, or 20% increase of FTO muscle fiber diameter by 4 weeks after administration; (g) at least a 5%, 10%, 15%, 20%, 25%, 30%, or 35% decrease of FTG muscle fiber number per mm² by 4 weeks after administration; and (h) at least a 5%, 10%, 15%, 20%, or 25% increase of FTG muscle fiber diameter by 4 weeks after administration.

The any of the uses of a therapeutically effective amount of any of the disclosed rAAV or a composition provided, after treatment with the medicament, the heart muscle of the subject shows no, minimum or low calpain 3 protein expressed from the disclosed or disclosed composition.

### BRIEF DESCRIPTION OF THE DRAWINGS

FIGS. 1A-1F show that gene therapy restored impaired regeneration in CAPN3-KO muscle. Schematic diagram of single-stranded AAV9.CAPN3 rAAV is shown in FIG. 1A. In between the 5′ and 3′ single strand ITRs (inverted terminal repeats), the muscle creatine kinase (MCK) promoter (563 bp) drives the expression of CAPN3 open reading frame (2466 bp). Also labeled is polyadenylation site (Poly A, 53 bp). Tibialis anterior (TA) muscles from CAPN3-KO mice were first injected with CTX, and 2 weeks later with 1×10¹¹ vg of AAV.CAPN3 to left TA (FIG. 1B) or PBS to right TA (FIG. 1C). Four weeks after rAAV injection, the muscle diameter increased and the lobulated fibers were less common compared to the untreated CAPN3-KO muscle. In FIG. 1D, lobulated fibers with a pattern of subsarcolemmal organelle, mitochondria distribution (arrows) suggest partial myotube fusion in the untreated CAPN3-KO muscle at higher magnification. Scale bar=20 μm for B-D. In FIG. 1E, the muscle fiber size distribution histograms (mean±SEM/mm² area; derived from 3 mice in each group) of the treated and untreated TA muscle from CAPN3-KO mice show a shift to larger diameter fibers with the treatment and an increase in the small diameter subpopulation present in the untreated group. In FIG. 1F, the Slow twiTch Oxidative (STO) fiber size distribution histograms show a larger number of small fibers (e.g., fiber diameters equal to or less than 30 μm) in the untreated CAPN3-KO muscle as compared to treated CAPN3-KO muscle.

FIG. 2 shows a schematic diagram of the rAAV of this disclosure, named as “AAVrh.74.tMCK.CAPN3.”

FIGS. 3A and 3B provide Western Blot (panel A) and RT-PCR (panel B) data after AAVrh.74.tMCK.CAPN3 administration via intramuscular injection (1E11 vg) and systemic injection (3E12 vg and 6E12 vg). This data was compared with normal human muscle lysate (Gel load of 60% total protein as compared to mouse lysates) and untreated CAPN3-KO mice.

FIG. 4 provides representative images of SDH-stained tissue sections of CAPN3 KO (AAV.hCAPN3 gene injected and untreated) and wild type (WT) TA muscles. Mean fiber size of slow twitch oxidative (STO, dark), fast twitch oxidative (FTO, intermediate) and fast twitch glycolytic (FTG, light) fibers appeared normalized towards WT values in the TA muscle of mice treated with AAVrh.74.tMCK.CAPN3. Fiber type sizes with and without treatment are illustrated in the Table 4.

FIG. 5 provides relative CAPN3 protein expression levels in WT (Z18-14) and TA muscles from the low dose cohort (3E12 vg, Z18-13, Z18-15, Z18-16, Z18-17, Z18-18), and gastrocnemius (gastroc), heart, quadriceps, tibialis anterior (TA) and triceps from the high dose cohort (6E12 vg, Z18-20, Z18-21, Z18-23, Z18-24, Z18-22) are shown (UT: untreated).

FIG. 6 provides AAVrh74.tMCK.hCAPN3 vector copies/ug genomic DNA in 6E12 vector genome systemic high dose cohort in the following muscles: quadriceps (quad), heart, tibialis anterior (TA), gastrocnemius (gastroc) triceps, and the liver.

FIG. 7 provides the mean fiber diameters of slow twitch oxidative (STO, dark), fast twitch oxidative (FTO, intermediate) and fast twitch glycolytic (FTG, light) fibers from left TA muscle following systemic administration of AAVrh.74.tMCK.CAPN3 at 3E12 and 6E12 vg. Data from untreated CAPN3KO and WT mice was included.

FIGS. 8A and 8B provides the data for the run-to-exhaustion test. FIG. 8A provides data for the low dose cohort, which received 3E12 vg of AAVrh.74.tMCK.CAPN3, and the high dose cohort, which received 6E12 vg of AAVrh.74.tMCK.CAPN3 4 weeks after systemic administration. Treated CAPN3 KO mice performed better on Run-to-Exhaustion test compared to untreated counterparts. FIG. 8B provides data for the high dose cohort, in which the mice were tested 20-24 weeks after systemic administration of 6E12 vg of AAVrh.74.tMCK.CAPN3 (n=5) and untreated counterparts (n-16)

FIG. 9 provides hematoxylin & cosin (H&E) stained fresh frozen sections of the left ventricles from representative heart samples of CAPN3 KO mice at 4 weeks post-systemic injection of the AAVrh7.4.tMCK.hCAPN3 vector at 3E12 vg and 6E12 vg doses with matching untreated controls.

FIG. 10 provides Western blot analysis of the cardiac tissues from the high dose cohort (which received 6E12 vg of AAVrh7.4.tMCK.hCAPN3. This analysis showed no or minimum detectable calpain 3 protein in the heart of the treated animal. Animal identification numbers Z18-19 and 22 represent the lysates from the untreated CAPN3 KO mice.

### DETAILED DESCRIPTION

Recombinant AAVs (rAA Vs) provided herein comprise a polynucleotide that comprises a first AAV inverted terminal repeat (ITR), a promoter, a nucleotide sequence encoding a protein with calpain 3 (CAPN3) activity and a second AAV ITR. In one embodiment, the nucleotide encodes CAPN3. Embodiments include, but are not limited to, an rAAV comprising a nucleotide sequence encoding CAPN3 or a protein with CAPN3 activity, wherein the nucleotide sequence is at least 65%, at least 70%, at least 75%, at least 80%, 81%, 82%, 83%, 84%, 85%, 86%, 87%, 88%, or 89% identical to the nucleotide sequence of SEQ ID NO: 2. Additional embodiments include, but are also not limited to, rAAV comprising a nucleotide sequence at least 90%, 91%, 92%, 93%, 94%, 95%, 96%, 97%, 98%, 99% identical to the nucleotide sequence set forth in SEQ ID NO: 2 and encodes a polypeptide with a CAPN3 proteolytic activity. The CAPN3 proteolytic activity is understood in the art as the activity of proteolyzing potential substrates such as fodrin and HSP60, and/or to the activity of autolytically self-cleaving. Thus, as used herein, the term “a protein with calpain 3 (CAPN3) activity” refers to a protein with CAPN3 proteolytic activity, which includes but is not limited to the activity of proteolyzing substrates such as fodrin and HSP60, and/or to the activity of autolytically self-cleaving. The protein with CAPN3 activity can have the full or partial activity of a full length calpain 3 protein. In one embodiment, the protein with CAPN3 activity has at least 60%, 70%, 80%, 90%, 95%, or 99% of activity of a full length CAPN3 protein. In another embodiment, the protein with CAPN3 activity comprises an amino acid sequence with at least 90%, 91%, 92%, 93%, 94%, 95%, 96%, 97%, 98%, or 99% identical to SEQ ID NO: 7.

In some embodiments, the nucleotide sequence encoding the protein with CAPN3 activity comprises a sequence of SEQ ID NO: 2. In another embodiment, the protein with CAPN3 activity comprises an amino acid sequence with at least 90%, 91%, 92%, 93%, 94%, 95%, 96%, 97%, 98%, or 99% identical to SEQ ID NO: 7. In another embodiment, the protein with CAPN3 activity comprises the amino acid sequence of SEQ ID NO: 7. In another embodiment, the polynucleotide of the rAAV comprises a sequence which is at least 90%, 91%, 92%, 93%, 94%, 95%, 96%, 97%, 98%, or 99% identical to SEQ ID NO: 1. In another embodiment, the polynucleotide comprises a sequence at least 95% identical to SEQ ID NO: 1. In one embodiment, the polynucleotide comprises the sequence of SEQ ID NO: 1.

In another aspect, described herein is a recombinant AAV comprising a nucleotide sequence that encodes a protein with CAPN3 activity and/or that comprises a nucleotide sequence that hybridizes under stringent conditions to the nucleic acid sequence of SEQ ID NO: 2, or the complement thereof. The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015M sodium citrate, and 50% formamide at 42° C. See Sambrook et al., Molecular Cloning: A Laboratory Manual, 2nd Ed., Cold Spring Harbor Laboratory, (Cold Spring Harbor, N.Y. 1989).

In recombinant genomes described herein, the CAPN3 polynucleotide is operatively linked to transcriptional control elements (including, but not limited to, promoters, enhancers and/or introns), specifically transcriptional control elements functional in target cells of interest. For example, various embodiment provide methods of transducing muscle cells using muscle-specific transcriptional control elements, including, but not limited to, those derived from the actin and myosin gene families, such as from the myoD gene family [See Weintraub et al., Science, 251: 761-766 (1991)], the myocyte-specific enhancer binding factor MEF-2 [Cserjesi and Olson, Mol Cell Biol, 11: 4854-4862 (1991)], control elements derived from the human skeletal actin gene [Muscat et al., Mol Cell Biol, 7: 4089-4099 (1987)], muscle creatine kinase sequence elements [See Johnson et al., Mol Cell Biol, 9:3393-3399 (1989)] and the murine creatine kinase enhancer (mCK) element, control elements derived from the skeletal fast-twitch troponin C gene, the slow-twitch cardiac troponin C gene and the slow-twitch troponin I gene: hypozia-inducible nuclear factors [Semenza et al., Proc Natl Acad Sci USA, 88: 5680-5684 (1991)], steroid-inducible elements and promoters including the glucocorticoid response element (GRE) [See Mader and White, Proc. Natl. Acad. Sci. USA, 90: 5603-5607 (1993)], the tMCK promoter [see Wang et al., Gene Therapy, 15: 1489-1499 (2008)], the CK6 promoter [see Wang et al., supra] and other control elements. In one embodiment, the nucleotide sequence encoding a protein with calpain 3 (CAPN3) activity is operably linked to a muscle-specific promoter. In one embodiment, the muscle-specific promoter comprises one or more of a human skeletal actin gene element, a cardiac actin gene element, a desmin promoter, a skeletal alpha-actin (ASKA) promoter, a troponin I (TNNI2) promoter, a myocyte-specific enhancer binding factor mef binding element, a muscle creatine kinase (MCK) promoter, a truncated MCK (tMCK) promoter, a myosin heavy chain (MHC) promoter, a hybrid a-myosin heavy chain enhancer-/MCK enhancer-promoter (MHCK7) promoter, a C5-12 promoter, a murine creatine kinase enhancer element, a skeletal fast-twitch troponin c gene element, a slow-twitch cardiac troponin c gene element, a slow-twitch troponin i gene element, hypoxia-inducible nuclear factor (HIF)-response element (HRE), a steroid-inducible element, a glucocorticoid response element (gre). In another embodiment, the muscle-specific promoter is an MCK promoter, a tMCK promoter, or an MHCK7 promoter. In some embodiments, the muscle-specific promoter is tMCK that comprises a nucleotide sequence of SEQ ID NO: 3.

Previous studies showed that expression of CAPN3 driven by desmin promoter resulted in cardiotoxicity. In follow up studies, selective skeletal muscle expression of the gene eliminated the cardiac defects. The AAV genomes disclosed herein comprise a muscle specific promoter, tMCK to restrict CAPN3 expression to the skeletal muscle and showed no cardiac toxicity following systemic delivery of the virus at 6E12 vg (twice the proposed initial high dose) 4 weeks after gene injection.

The rAAV genomes described herein lack AAV rep and cap DNA. rAAV genomes provided comprise a CAPN3 polynucleotide as described above and one or more AAV ITRs flanking the polynucleotide. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV rh. 74 and AAV rh. 10. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art. To promote skeletal muscle specific expression, AAVI, AAV5, AAV6, AAV8 or AAV9 may be used.

DNA plasmids provided comprise rAAV genomes. The DNA plasmids are transferred to cells permissible for infection with a helper virus of AAV (including, but not limited to, adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV ITRs and rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV rh. 10 and AAV rh.74. Production of pseudotyped rAAV is disclosed in, for example, WO 01/83692 which is incorporated by reference herein in its entirety. Thus, in one embodiment, the rAAV comprises one or more of AAV-1, AAV-2, AAV-3, AAV-4, AAV-5, AAV-6, AAV-7, AAV-8, AAV-9, AAV-10, AAV-11, AAV-12, AAV-13, AAV rh. 74 or AAV rh. 10 capsid proteins. In another embodiment, the rAAV comprises an AAV rh.74 capsid protein or an AAV9 capsid protein.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing [Samulski et al., Proc. Natl. Acad. S6. USA, 79:2077-2081 (1982)], addition of synthetic linkers containing restriction endonuclease cleavage sites [Laughlin et al., Gene, 23:65-73 (1983)] or by direct, blunt-end ligation [Senapathy & Carter, J. Biol. Chem., 259:4661-4666 (1984)]. The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, Current Opinions in Biotechnology, 1533-1539 (1992); and Muzyczka, Curr. Topics in Microbial. and Immunol., 158:97-129 (1992). Various approaches are described in Ratschin et al., Mol. Cell. Biol., 4:2072 (1984); Hermonat et al., Proc. Natl. Acad. Sci. USA, 81:6466 (1984); Tratschin et al., Mol. Cell. Biol., 5:3251 (1985); Mclaughlin et al., J. Virol., 62:1963 (1988); Lebkowski et al., Mol. Cell. Biol., 7:349 (1988); Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine, 13:1244-1250 (1995); Paul et al., Human Gene Therapy, 4:609-615 (1993); Clark et al., Gene Therapy 3:1124-1132 (1996); U.S. Pat. No. 5,786,211; U.S. Pat. No. 5,871,982; U.S. Pat. No. 6,258,595; and McCarty, Mol. Ther., 16(10): 1648-1656 (2008). The foregoing documents are hereby incorporated by reference in their entirety herein, with particular emphasis on those sections of the documents relating to rAAV production.

Thus packaging cells are provided that produce infectious rAAV. In one embodiment, packaging cells may be stably transformed cancer cells such as HeLa cells, and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Recombinant AAV provided herein are thus replication-deficient, infectious, encapsidated viral particles which comprise a recombinant genome. Examples include, but are not limited to, a rAAV including a genome comprising the sequence set out in SEQ ID NO: 1 encoding CAPN3, a rAAV including a genome consisting essentially of the sequence set out in SEQ ID NO: 1 encoding CAPN3, and a rAAV (named “AAVrh.74.tMCK.CAPN3”) including a genome consisting of the sequence set out in SEQ ID NO: 1 encoding CAPN3. The genomes of the rAAV lack AAV rep and cap DNA, that is, there is no AAV rep or cap DNA between the ITRs of the rAAV genome.

The sequence of the AAVrh. 74.tMCK.CAPN3 sequence is set out in SEQ ID NO: 1, in which an AAV2 ITR spans nucleotides 1-128, the tMCK promoter spans nucleotides 165-884, a chimeric intron spans nucleotides 937-1069, a Kozak Sequence spans nucleotides 1101-1106, the CAPN3 polynucleotide spans nucleotides 1107-3572, a poly A signal spans nucleotides 3581-3780, and a second AAV2 ITR spans nucleotides 3850-3977.

The rAAV may be purified by methods known in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69:427-443 (2002); U.S. Pat. No. 6,566,118; and WO 98/09657.

In another embodiment, compositions comprising rAAV described herein are provided. Compositions provided comprise rAAV in a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-formig counterions such as sodium; and/or nonionic surfactants such as Tween, pluronics or polyethylene glycol (PEG).

Titers of rAAV to be administered in methods described herein can vary depending, for example, on the particular rAAV, the mode of administration, the treatment goal, the individual, and the cell type(s) being targeted, and may be determined by methods standard in the art. Titers of rAAV may range from about 1×10¹⁰, about 1×10¹¹, about 1×10¹², about 1×10¹³, to about 1×10¹⁴, or more DNase resistant particles (DRPs) per ml. Dosages may also be expressed in units of viral genomes (vg). Exemplary disclosed doses include 1E11 vg, 3E12 vg and 6E12 vg.

Methods of transducing a target cell such as a muscle cell with rAAV, in vivo or in vitro, are contemplated herein. The in vivo methods comprise the step of administering an effective dose, or effective multiple doses, of a composition comprising a rAAV provided herein to subject (e.g., an animal including but not limited to a human patient) in need thereof. If the dose is administered prior to development of a disorder/disease, the administration is prophylactic. If the dose is administered after the development of a disorder/disease, the administration is therapeutic. An effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. In comparison to the subject before treatment, methods herein result in one or more of: an increased muscle fiber diameter, a decreased number of small lobulated muscle fibers, a decreased number of fibers with internal nuclei, a decreased endomysial connective tissue content, correction of muscle atrophy, and an increased muscle force generation. In one embodiment, the muscle fiber comprises one or more of slow twitch oxidative (STO) muscle fiber, fast twitch oxidative (FTO) muscle fiber, and fast twitch glycolytic (FTG) fiber. In one embodiment, the treatment results in one or more of (a) at least a 5%, 10%, 15%, 20%, 25%, 30%, or 35%, or 40% decrease of total muscle fiber number per mm² by 4 weeks after administration; (b) at least a 5%, 10%, 15%, 20%, or 25% increase of muscle fiber diameter by 4 weeks after administration; (c) at least a 5%, 10%, 15%, 20%, 25%, 30%, 35%, or 42% decrease of STO muscle fiber number per mm² by 4 weeks after administration; (d) at least a 5%, 10%, 15%, 20%, or 25% increase of STO muscle fiber diameter by 4 weeks after administration; (e) at least a 5%, 10%, 15%, or 20% decrease of FTO muscle fiber number per mm² by 4 weeks after administration; (f) at least a 5%, 10%, 15%, or 20% increase of FTO muscle fiber diameter by 4 weeks after administration; (g) at least a 5%, 10%, 15%, 20%, 25%, 30%, or 35% decrease of FTG muscle fiber number per mm² by 4 weeks after administration; and (h) at least a 5%, 10%, 15%, 20%, or 25% increase of FTG muscle fiber diameter by 4 weeks after administration. The method of this disclosure, in one embodiment, leads to no, minimum or low calpain 3 protein expressed from the rAAV in the heart muscle of the subject administered with the rAAV.

Assays to examine these results are understood in the art and/or are described in the examples herein. Use of the methods described herein to prevent or treat disorders/diseases (e.g., muscular dystrophies) caused by defects in CAPN3 activity or defects in expression of CAPN3 is contemplated. LGMD2A is an example of a disease contemplated for prevention or treatment according to the methods.

Combination therapies are also contemplated. Combination as used herein includes both simultaneous treatment or sequential treatments. Combinations of methods described herein with standard medical treatments (e.g., corticosteroids) are specifically contemplated, as are combinations with novel therapies.

Administration of an effective dose of the compositions may be by routes standard in the art including, but not limited to, intramuscular, parenteral, intravenous, intrathecal, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) may be chosen and/or matched by those skilled in the art taking into account the infection and/or disease state being treated and the target cells/tissue(s) that are to express the CAPN3. In one embodiment, the rAAV is administered by intramuscular injection, intravenous injection, intraperitoneal injection, subcutaneous injection, epicutaneous administration, intravaginal injection, intradermal administration, or nasal administration. In another embodiment, the rAAV is administered by intramuscular injection or intravenous injection.

In particular, actual administration of rAAV described herein may be accomplished by using any physical method that will transport the rAAV recombinant vector into the target tissue of an animal. Administration includes, but is not limited to, injection into muscle, the bloodstream, and/or directly into the liver. Simply resuspending a rAAV in phosphate buffered saline has been demonstrated to be sufficient to provide a vehicle useful for muscle tissue expression, and there are no known restrictions on the carriers or other components that can be co-administered with the rAAV. Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the methods. The rAAV can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for systemic (e.g., intravenous) injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the methods of preparation, in some embodiments, comprises vacuum drying and/or the freeze drying technique, each of which can yield a powder of the active ingredient plus any additional desired ingredient from the previously sterile-filtered solution thereof.

Transduction with rAAV may also be carried out in vitro. In one embodiment, desired target muscle cells are removed from the subject, transduced with rAAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one embodiment, cells can be transduced in vitro by combining rAAV with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with rAAV by methods described herein results in sustained expression of CAPN3 or a protein with CAPN3 activity. Methods are thus provided for administering rAAV which expresses CAPN3 or a protein with CAPN3 activity to a subject, preferably a human being. The subject of this disclosure includes but is not limited to human, a dog, a cat, a horse, a cow, a pig, a sheep, a goat, a chicken, a rodent (e.g., rats and mice), and a primate. These methods include transducing tissues (including, but not limited to, tissues such as muscle, organs such as liver and brain, and glands such as salivary glands) with one or more rAAV described herein.

Muscle tissue is an attractive target for in vivo DNA delivery, because it is not a vital organ and is easy to access. The methods herein provide sustained expression of CAPN3 from transduced muscle cells.

By “muscle cell,” “muscle fiber,” or “muscle tissue” is meant a cell or group of cells derived from muscle of any kind [for example, skeletal muscle and smooth muscle (e.g., from the digestive tract, urinary bladder, blood vessels or cardiac tissue)]. Such muscle cells may be differentiated or undifferentiated, such as myoblasts, myocytes, myotubes, cardiomyocytes and cardiomyoblasts.

The term “transduction” is used to refer to the administration/delivery of CAPN3 to a recipient cell either in vivo or in vitro, via a rAAV described resulting in expression of CAPN3 by the recipient cell.

Thus, methods are provided of administering an effective dose (or doses, administered essentially simultaneously or doses given at intervals) of rAAV that encode CAPN3 to a subject in need thereof.

As noted above, the methods described herein result in the subject, in comparison to the subject before treatment, one or more of: increased muscle fiber diameter, decreased number of small lobulated slow twitch oxidative (STO) muscle fibers, decreased number of fibers with internal nuclei, decreased endomysial connective tissue content, correction of muscle atrophy, and increased muscle force generation.

### EXAMPLES

Aspects and embodiments are illustrated by the following examples. Example 1 describes production of AAV9.MCK.CAPN3. Example 2 describes intramuscular administration of AAV9.MCK.CAPN3. Example 3 describes production of AAVrh.74.tMCK.CAPN3. Example 4 describes intramuscular administration of AAVrh.74.tMCK.CAPN3. Example 5 describes intravenous administration of AAVrh.74.tMCK.CAPN3. Example 6 describes end point studies. Example 7 describes toxicology and biodistribution studies. Example 8 describes in vivo biopotency testing following intramuscular injection. Example 9 describes in vivo biopotency testing following systemic injection. Example 10 describes assessment of systemic AAVrh.74.tMCK.CAPN3 gene delivery. Example 11 describes assessment of cardiac toxicity following systemic injection of AAVrh.74.tMCK.CAPN3 vector. Example 12 describes in vivo physiological analysis.

### Example 1

### Production of AAV9.MCK.CAPN3

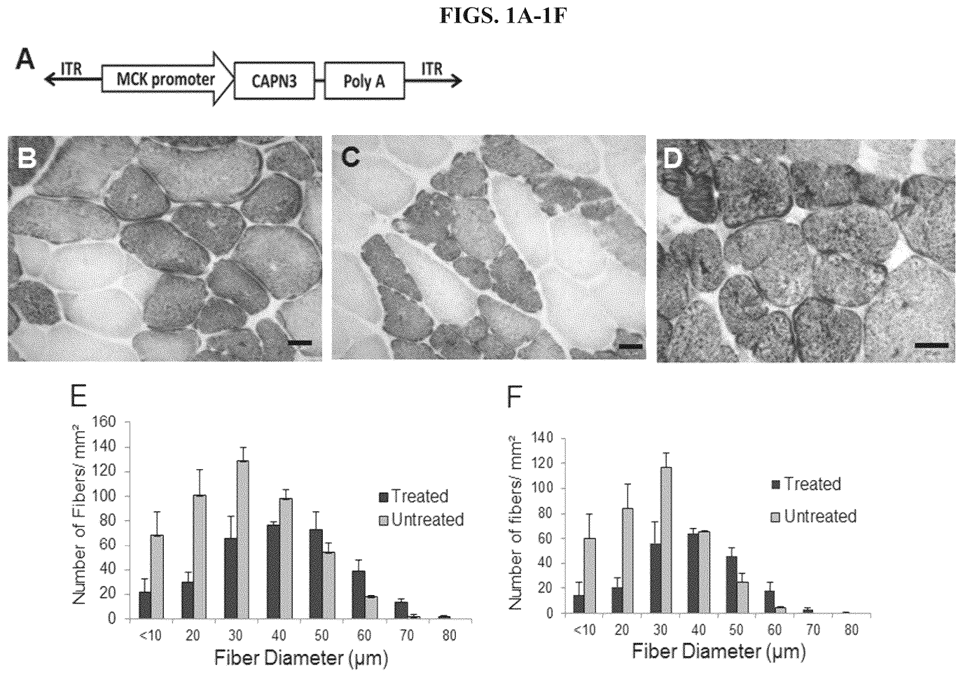

An AAV vector (named AAV.CAPN3) carrying the CAPN3 gene under the muscle specific MCK promoter (FIG. 1A) was produced. A DNA including the open reading frame of mouse ('APN3 (NM_007601.3) between two Not1 restriction sites was synthesized by Eurofin Genomics, USA, and then subcloned into a single strand AAV.MCK (muscle creatine kinase) vector previously described in Rodino-Klapac et al., Journal of Translational Medicine, 5:45-55 (2007)]. rAAV vectors were produced by a modified cross-packaging approach whereby the AAV type 2 vector genome can be packaged into multiple AAV capsid serotypes. [Rabinowitz et al., J Virol. 76 (2): 791-801 (2002)]. Production was accomplished using a standard three-plasmid DNA/CaPO4 precipitation method using HEK293 cells. 293 cells were maintained in DMEM supplemented with 10% fetal bovine serum (FBS) and penicillin and streptomycin. The production plasmids were: (i) pAAV.MCK.microdys, (ii) rep2-capX modified AAV helper plasmids encoding cap serotypes 1, 6, or an 8-like isolate, and (iii) an adenovirus type 5 helper plasmid (pAdhelper) expressing adenovirus E2A, E4 ORF6, and VA I/II RNA genes. To allow comparisons between serotypes, a quantitative PCR-based titration method was used to determine an encapsidated vector genome (vg) titer utilizing a Prism 7500 Taqman detector system (PE Applied Biosystems). [Clark et al., Hum Gene Ther. 10 (6): 1031-1039 (1999)] The primer and fluorescent probe targeted the MCK promoter and were as follows: MCK forward primer, 5-CCCGAGATGCCTGGTTATAATT-3 (SEQ ID NO: 4); MCK reverse primer, 5-GCTCAGGCAGCAGGTGTTG-3 (SEQ ID NO: 5); and MCK probe, 5-FAM-CCAGACATGTGGCTGCTCCCCC-TAMRA-3 (SEQ ID NO: 6). The final titer (vg ml 1) was determined by quantitative reverse transcriptase PCR using the specific primers and probes for MCK promoter utilizing a Prism 7500 Real-time detector system (PE Applied Biosystems, Grand Island, NY, USA). Aliquoted viruses were kept at −80° C. until use.

### Example 2

### Intramuscular Administration of AAV9.MCK.CAPN3

To demonstrate if WT CAPN3 can restore the impaired regeneration process in CAPN3 knockout (CAPN3-KO) mice, TA muscles from CAPN3-KO mice (n=4) [Kramerova et al., Hum Mol Genet 13(13):1373-1388 (2004)]] under anesthesia were first injected with 30 μl CTX, and 2 weeks later were transduced to express wild type CAPN3 using AAV9.MCK.CAPN3 at 1×10¹¹ vg in 20 μl volume via intramuscular injection. TA muscles from another cohort of CAPN3-KO (n=4), served as controls received the same volume of PBS 2 weeks post-CTX injection.

Mice were killed at 6 weeks post-CTX injection, and TA muscles were removed and processed for cryostat sectioning. Twelve μm thick cross sections were first stained with H&E for routine histopathological evaluation; muscle fiber type specific diameter measurements were obtained from SDH stained cross sections of the TA from 3 mice in each group. Three random images of the TA (per section per animal) was photographed at X20 magnification and the fiber diameter measurements and fiber type specific histograms were generated.

Succinic dehydrogenase (SDH) enzyme histochemistry was used to assess metabolic fiber type differentiation [slow twitch oxidative (STO), fast twitch oxidative (FTO) and fast twitch glycolytic (FTG)]. Muscle fiber type specific diameter measurements were obtained using 12 μm thick-SDH stained cross sections at 4 and 12 weeks after final cardiotoxin injection. Three images, each representing three distinct zones of the gastrocnemius muscle (a deep zone predominantly composed of STO, intermediate zone showing a checkerboard appearance of STO and FTO or FTG and the superficial zone predominantly composed of FTG fibers) along the midline axis (per section per animal) was photographed at X20 magnification using an Olympus BX41 microscope and SPOT camera (Olympus BX61, Japan). This approach was chosen to capture the alterations in the oxidative state of fibers in each zone in response to metabolic changes during regeneration. Diameters of dark (STO), intermediate (FTO) and light (FTG) fibers were determined by measuring the shortest distance across the muscle fiber using Zeiss Axiovision LE4 software (v.4.8). The fiber diameter histograms were generated separately for STO; FTG and FTO were combined to represent the total fast twitch fiber population (FTG/O), derived from 3 animals and expressed as number per mm² of endomysial area (mean±SEM). The mean fiber diameter was derived from combining all 3 fiber types. An average of 900-1700 fibers were measured per group. TA muscles were used for assessment of fibrosis (see below)

Four weeks after AAV9.MCK.CAPN3 injection, a significant increase in muscle diameter with an apparent decrease of internal nuclei and far less number of small fibers with lobulated pattern was observed (FIG. 1B). The untreated CAPN3-KO muscle had 31.6% more fibers per mm² area, mostly composed of small and lobulated STO fibers indicating that the treatment improved myotube fusion, therefore decreased individual small fiber number per unit area (FIGS. 1C and 1D; Table 1).

TABLE 1
Tibialis anterior muscle fiber size

| | | | AAV.CAPN3- | |
|---|---|---|---|---|
| | Untreated | | treated | AAV.CAPN3- |
| | Number | Untreated | Number | treated |
| | per mm² | Diameter | per mm² | Diameter |
| STO | 355 | 32.72 ± 0.4 | 233 | 39.81 ± 0.6* |
| FTG/O | 116 | 44.26 ± 0.9 | 99 | 50.40 ± 1.2* |
| All | 471 | 35.55 ± 0.4 | 322 | 43.08 ± 0.6* |
| fibers | | | | |

*p < 0.0001 compared to same wild type parameter

The fiber size distribution histograms of the treated TA muscle showed a shift to larger diameter fibers with treatment and the excessive number of small fibers in the untreated CAPN3-KO control muscle are of STO histochemical fiber type (FIGS. 1E and 1F). Collectively, these findings show that CAPN3 replacement via gene therapy in the CAPN3-KO muscle rescued defective regeneration, evidenced by toward normalization of fiber size and a decrease in the number of STO fiber population.

### Example 3

### Production of AAVrh.74.tMCK.CAPN3

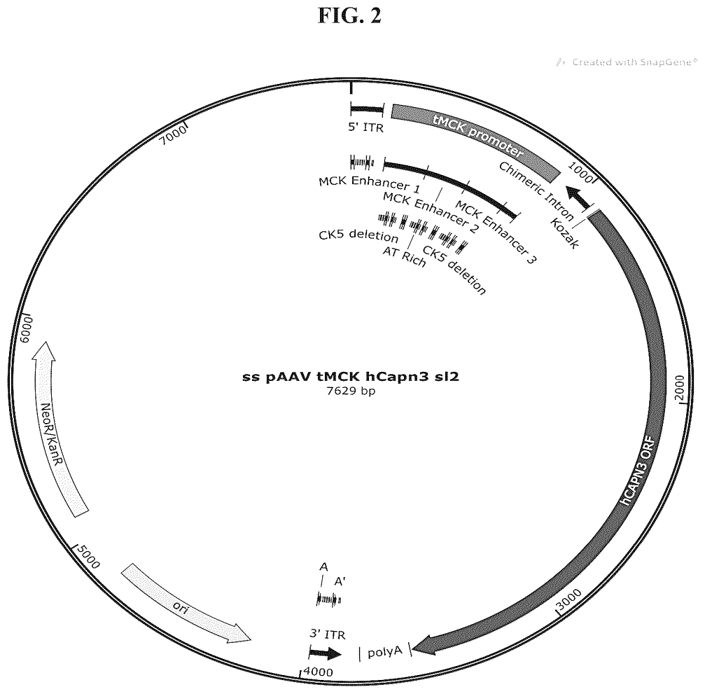

An AAV vector (named AAVrh74.tMCK.CAPN3) carrying the CAPN3 gene under a truncated muscle specific MCK promoter (tMCK promoter) was produced. A DNA including the open reading frame of mouse CAPN3 (NM_007601.3) between two Not1 restriction sites was synthesized by Eurofin Genomics, USA, and then inserted in an AAV production plasmid. A map of the plasmid is shown in FIG. 2.

rAAV vectors were then produced by the approach described in Example 1.

### Example 4

### Intravenous Administration of AAVrh.74.tMCK.CAPN3

CAPN3-KO mice, 6 months of age, received AAVrh.74.tMCK.CAPN3 at low (3×10¹² vg) and high doses (6×10¹² vg) via injection into tail vein. The mice were killed at 20 weeks post gene injection for endpoint studies. Age matched vehicle treated CAPN3-KO mice served as controls.

TABLE 2
Treatment cohorts

| | | | | | | Age |
|---|---|---|---|---|---|---|
| | | Total | Age at | | | at |
| | | # of | start of | Treatment Dose | Treatment | End |
| Cohorts | Treatment | mice | treatment | (AAVrh.74.tMCK.CAPN3) | Duration | Point |
| CAPN3- | | 40 | | | | |
| KO | | | | | | |
| Low | AAV.CAPN3 | 8 | 24 wks | 3e12 vg in 300 μl saline, i.v. | 20 wks | 44 wks |
| dose | | | | | | |
| | Saline | 8 | 24 wks | | 20 wks | 44 wks |
| | treatment | | | | | |
| High | AAV.CAPN3 | 8 | 24 wks | 6e12 vg in 300 μl saline, i.v. | 20 wks | 44 wks |
| Dose | | | | | | |
| | Saline | 8 | 24 wks | | 20 wks | 44 wks |
| | treatment | | | | | |
| Wild | Saline | 8 | 24 wks | | 20 wks | 44 wks |
| type | treatment | | | | | |
| Controls | | | | | | |

End point studies performed as described in Example 7 below include muscle physiology (TA force generation or in vivo muscle contractibility assay, and protection from eccentric contractions), muscle histopathology, hCAPN3 detection using qPCR, and Western blot analysis.

### Example 5

### Intramuscular Administration of AAVrh.74.tMCK.CAPN3

Regenerative responses are measured in old and young CAPN3-KO muscle to cardiotoxin (CTX)-induced synchronized necrosis following the introduction of CAPN3 into regenerating muscle via rAAV treatment.

In cohorts of young (at 2 months of age) and old mice (at 6 months of age), CTX is injected into both TA muscles to induce synchronized necrosis 2 weeks prior to rAAV injection to the left TA muscle. AAVrh.74.tMCK.CAPN3 at 1×10¹¹ vg in 20 μl volume is administered via intramuscular injection. Endpoint studies are performed at 8 weeks post gene transfer (at 1×10¹¹ vg dose with efficacy established in our previous studies) to assess the correction of regeneration defect by comparing quantitative histology and physiological outcomes from the left TA to untreated right TA.

TABLE 3
Treatment cohorts

| | | | CTX inj-bilateral | Age at | | | Age |
|---|---|---|---|---|---|---|---|
| | | Total | TA muscle; | start of | Treatment Dose | | at |
| | | # of | age/delivery | gene | (AAVrh.74.tMCK.CAPN3) | Treatment | End |
| Cohorts | Treatment | mice | route/dose | therapy | Left TA | Duration | Point |
| CAPN3- | CTX + | 16 | | | | | |
| KO | AAV.CAPN3 | | | | | | |
| Young | | 8 | 6 wks/i.m./30 μl | 8 wks | 1e11 vg in 30 μl PBS, i.m. | 8 wks | 16 wks |
| Old | | 8 | 22 wks/i.m./30 μl | 24 wks | 1e11 vg in 30 μl PBS, i.m. | 8 wks | 32 wks |
| Wild | CTX only | 18 | | | | | |
| type | | | | | | | |
| Young | | 8 | 6 wks/i.m./30 μl | 8 wks | 30 μl PBS | 8 wks | 16 wks |
| Old | | 8 | 22 wks/i.m./30 μl | 24 wks | 30 μl PBS | 8 wks | 32 wks |

Eight weeks post-rAAV injection, end point studies carried out as described in Example 6 below include muscle physiology (TA force generation and protection from eccentric contractions), quantitative muscle histopathology, hCAPN3 detection using qPCR and western blot analysis.

### Example 6

### End Point Studies

### TA Force Generation and Protection From Eccentric Contractions

A protocol to assess functional outcomes in the TA muscle is performed on muscles extracted from mice [Wein et al., Nature Medicine, 20(9):992-1000 (2014)]. Mice are anesthetized using ketamine/xylazine mixture. Using a dissecting scope, the hind limb skin is removed to expose the TA muscle and the patella. The distal TA tendon is dissected out and a double square knot is tied around the tendon with 4-0 suture as close to the muscle as possible, and the tendon is cut. The exposed muscle is constantly dampened with saline. Mice are then transferred to a thermal-controlled platform and maintained at 37 degrees. The knee is secured to the platform with a needle through the patella tendon, the distal TA tendon suture to the level arm of the force transducer (Aurora Scientific, Aurora, ON, Canada), and the foot is secured with tape. The TA muscle contractions are elicited by stimulating the sciatic nerve via bipolar platinum electrodes. Once the muscle is stabilized, the optimal length was determined by incrementally stretching the muscle until the maximum twitch force was achieved. After a 3 min rest period, the TA is stimulated at 50,100,150 and 200 Hz, allowing a 1 min rest period between each stimulus to determine maximum tetanic force. Muscle length is measured. Following a 5 min rest, the susceptibility of the TA muscle to contraction induced damage is assessed. After 500 ms of stimulation, the muscle is lengthened by 10% of the optimal length. This includes stimulating the muscle at 150 Hz for 700 ms. After the stimulation, the muscle is returned to the optimal length. The cycle is repeated every minute for a total of 10 cycles. Specific force is calculated by dividing the maximum tetanic force by the TA muscle cross-sectional area. After the eccentric contractions, the mice are then euthanized, and the TA muscle is dissected out, weighed and frozen for analysis. Analysis of the data is performed blindly but not randomly.

### In Vivo Muscle Contractibility Assay

This assay measures the aggregate torque produced by either the plantar or dorsiflexor muscles of the lower limb and is carried out using muscle physiology apparatus (Aurora Scientific, ON, Canada). The animal is anesthetized with isoflurane. Once the animal is anesthetized, the hair from the back and the hind limb will be removed as needed with clippers. If hair removal with clippers is insufficient, a thin layer of hair-removal cream (Nair) is applied, and the site thoroughly cleaned with warm water to prevent discomfort. The hindlimb to be measured is attached to the foot plate with adhesive tape. The limb is held rigid in a blunt clamp. Either the tibial or peroneal component of the sciatic nerve will be stimulated with two sterile, disposable 28 gauge monopolar electrodes inserted through the skin, subcutaneously near the nerve. Mouse temperature will be maintained by conductive thermoregulated heating pad (set at 37° C.) or radiant heat source and monitored by temperature probe.

### Histopathology

For histological analysis all muscles and organs are embedded in 7% gum tragacanth and flash frozen in liquid nitrogen cooled isopentane. Frozen sections (12 μm) are collected for immunohistochemistry and western blot analysis.

### Western Blot Analysis for Detection of Human CAPN3

CAPN3 protein quantification in mouse muscle tissues is assessed using a Western blotting method. The CAPN3 enzyme is resolved by sodium dodecyl sulfate polyacrylamide gel electrophoresis (SDS-PAGE) and migrates as a 94 kDa band with an autolytic product at approximately 60 kDa using Novocastra's clinical-grade antibody recognizing the N-terminus, NCL-CALP-12A2. Additionally, NCL-CALP-2C4 antibody recognizes this same CAPN3 molecular weight (94 kD), and an additional fragment (30 kD) in skeletal muscle; both antibodies are suitable for protein detection. A semi-quantitative measure of CAPN3 protein expression levels within the calpain-knockout mouse samples following delivery of the therapeutic rAAV vector is performed and compared with untreated controls.

### Quantitative Muscle Histology

Cross sections of TA and quad muscles from treated with AAVrh.74.tMCK.CAPN3 versus control uninjected, are stained with hematoxylin and eosin, and photographed using Zeiss Axiovision L4 software (4 random 20× images per section per animal). Fiber size diameters are compared between treated and controls.

### Statistical Analysis

Student's t-test or one-way ANOVA multiple comparison tests are performed where applicable.

### Example 7

### Toxicology/Biodistribution Studies

Toxicology/biodistribution studies are carried out using the established efficacious dose and one log higher dose. Toxicology studies are done by systemic (tail vein) delivery of rAAV to 6-8 week old CAPN3-KO mice including comparison to normal C57B16 normal mice. Cohorts of 6-10 mice are included and full necropsies are done using GLP-like methods.

Serum collected from blood samples is used for Clinical Chemistries: Alanine aminotransferase, Alkaline Phosphatase, Aspartate aminotransferase, Bilirubin (Total and Direct), Blood Urea nitrogen, Creatinine, Creatine Kinase, Glucose, and Total Protein.

A full necropsy is performed with a thorough and systematic examination and dissection of the animal viscera and carcass. The tissues/organs are collected include gonads, brain, spleen, kidneys, jejunum, colon, pancreas, heart, lung, stomach, liver, inguinal lymp nodes, spinal cord gastocnemius and quadriceps. Tissues/organs for histopathology studies are collected and fixed in 10% neutral buffered formalin (10% NBF), with the exception of all skeletal muscle specimens which are mounted on blocks with OCT, and flash frozen in liquid nitrogen-cooled methyl-butane for cryosections.

### Example 8

### In Vivo Biopotency Testing Following Intramuscular Injection

In vivo biopotency testing was carried out following intramuscular (IM) injection of AAVrh.74.tMCK.CAPN3 (1E11 vg) into the tibialis anterior (TA) muscle in CAPN3 KO mice (n=3) as described above in Example 5.

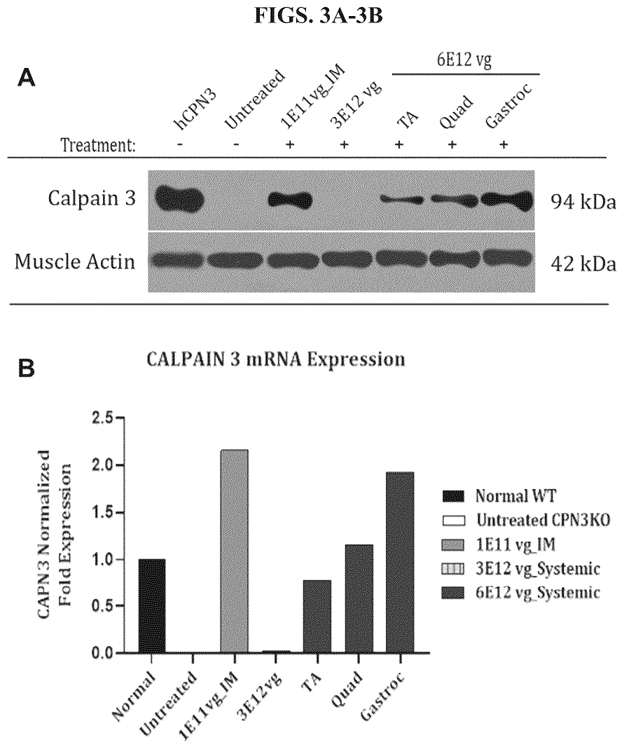

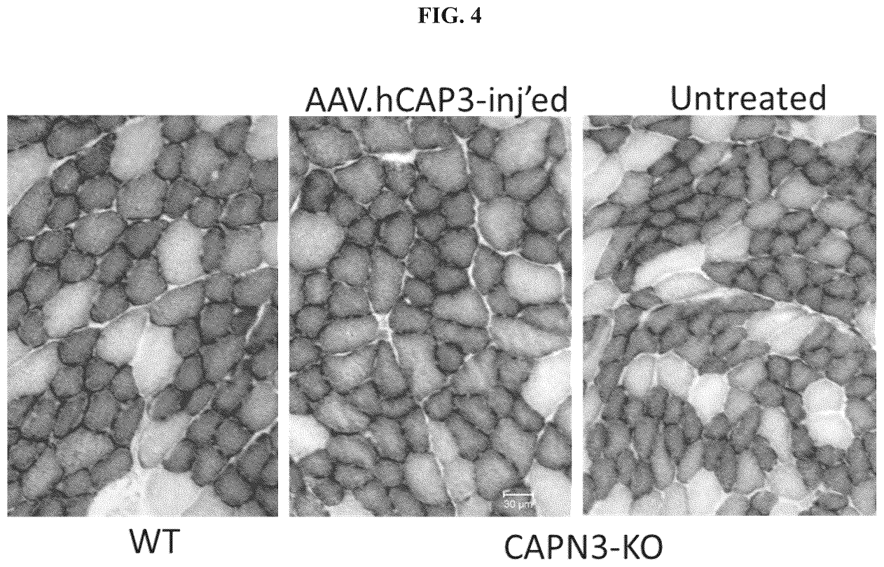

At 4 weeks post-administration, gene delivery was analyzed by reverse transcription quantitative PCR (RT-qPCR) and western blot analyses. For the Western blot analysis, samples corresponding to 50 μg of whole muscle protein extracts were separated on a 3-8% acrylamide, Tris-Acetate SDS gel and transferred to a PVDF membrane. Immunodetection was performed with a monoclonal antibody raised against s synthetic peptide containing AAs 1-19 of the human Calpain 3 sequence (Leica), and muscle specific actin antibody (Leica) as a loading control. FIG. 3A demonstrates that the presence of the 94 kD calpain 3 protein in the TA muscle after intramuscular injection. The RT-qPCR analysis demonstrated relative expression levels of human Calpain 3 gene 4 weeks post-gene transfer return to normalized levels as compared to WT mice (see FIG. 3B). Mouse GAPDH was used as a reference gene and WT C57BL/6 was used to calibrate the RT-qPCR data.

In addition, quantitative histopathological analysis was carried out after intramuscular administration. As shown in, the diameter of the TA muscle fiber of the treated CAPN3 KO mice was compared to that of the untreated control (ringer lactate injected TA) muscle. Mean fiber size of slow twitch oxidative (STO, dark), fast twitch oxidative (FTO, intermediate) and fast twitch glycolytic (FTG, light) fibers appeared normalized towards WT values in the AAV.hCAPN3 injected TA muscle. The quantification of the fiber type size is provided in Table 4 and illustrates an increase with treatment.

TABLE 4

| | WT (z18-14) | | Treated (z18-11) | | Untreated (z18-22 L) | |
|---|---|---|---|---|---|---|
| | number | diameter (μm) | number | diameter (μm) | number | diameter (μm) |
| STO | 246 | 28.06 ± 0.27 | 142 | 28.89 ± 0.32 | 240 | 25.57 ± 0.27 |
| FTO | 63 | 36.65 ± 0.53 | 86 | 36.71 ± 0.58 | 110 | 32.19 ± 0.48 |
| FTG | 82 | 42.55 ± 0.53 | 86 | 43.68 ± 0.66 | 128 | 35.49 ± 0.50 |
| All fiber | 391 | 32.45 ± 0.38 | 314 | 35.08 ± 0.45 | 478 | 29.75 ± 0.30 |

In summary, the in vivo biopotency testing following IM injection of the vector (1E11 vg) into tibialis anterior (TA) muscle in CAPN3 KO mice (n=2) demonstrated that 4 weeks post-gene delivery 1) RT-qPCR and western blot analyses showed expressions of CAPN3 transcripts and 94 kDa full-length calpain 3 protein and 2) histological analysis showed an increase in the muscle fiber diameter of TA compared to the control (Ringer's lactate injected TA) muscle.

### Example 9

### In Vivo Biopotency Testing Following Systemic Injection

In vivo biopotency testing was carried out following systemic injection of AAVrh.74.tMCK.CAPN3 (3E12 vg or 6E12 vg) via the tail vein of CAPN3-KO mice. The low dose CAPN3KO cohort (n=5; mice were denoted as Z18-13, Z18-15, Z18-16, Z18-17, Z18-18) received 3E12 vg in 300 μl Ringer's lactate. At 4 weeks post-gene injection, mice were evaluated for running fatigue by the run-to-exhaustion treadmill test and then euthanized for tissue collection. Muscles from upper and lower limbs (TA, gastrocnemius (GAS), quadriceps, triceps), heart, liver spleen, lung, ovaries and testicles were removed, and tissue samples were frozen in isopentane, and cooled in liquid nitrogen.

RT-qPCR CAPN3 expression was evaluated in TA muscles. For the 3E12 vg low dose, CAPN3 mRNA expression levels were low as observed by high CT values, >27. Western blot analysis showed undetectable corresponding protein bands. Even though low expression data was observed in this tissue for the low dose, both functional and histological benefits were demonstrated with the systemic administration of 3E12 vg.

Subsequently, a higher dose (6E12 vg) was systemically administered to investigate whether protein expression could be detected at a higher dose of vector delivery. The high dose cohort (mice denoted as Z18-20, Z18-21, Z18-23 and Z18-24) CAPN3-KO mice received 6E12 vg AAVrh7.4.tMCK.hCAPN3 vector (twice the dose used in the low dose cohort via systemic injection to the tail vein), and were euthanized 4 weeks post-injection. RT-qPCR showed variable levels of CAPN3 expression in the quad, triceps, GAS, TA and cardiac muscle.

To determine relative expression of the CAPN3 mRNA, muscle tissue samples were collected from CAPN3 KO mice treated with tMCK.hCAPN3 vector at the dose of 3E12 vg (low dose cohort 1) and 6E12 vg (high dose cohort 2). Total RNA was isolated from both cohorts and qPCR of CAPN3 vs. mouse GAPDH were assayed along with the previous samples from the cohort that received the vector via IM injection (1E11 vg; see above in Example 8).

The relative expression of CAPN3 was determined by the method below:

$$CT = {CT}_{CAPN 3} - {CT}_{m G A P D H}$$

$$ΔΔ CT = Δ C T - Δ C {T}_{C a l i b r a t o {r}^{*}}$$

$$Relative Expression of CAPN 3 = {2}^{- Δ Δ C T}$$

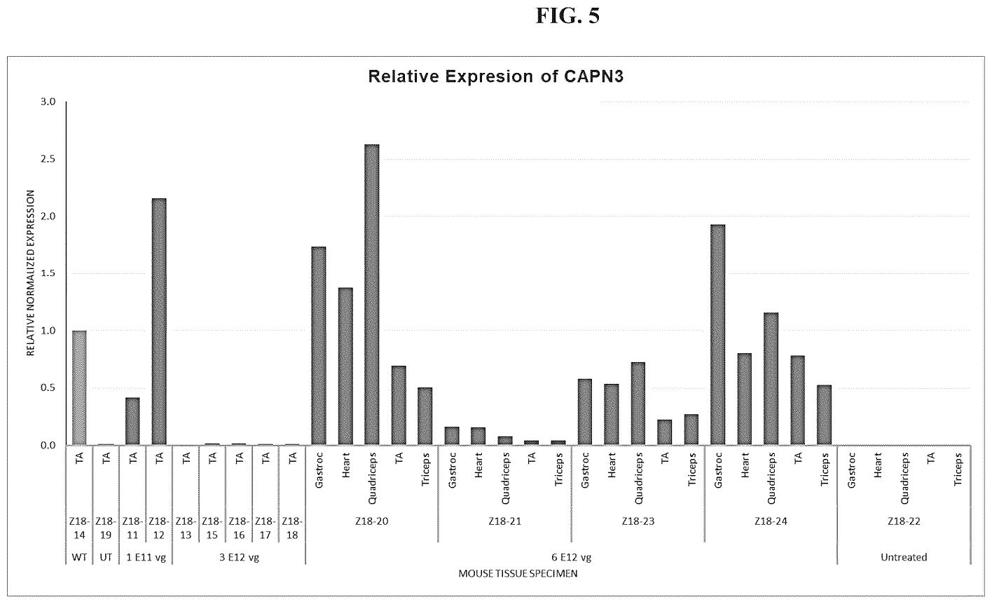

The relative expression of CAPN3 in each tissue and the original CT value were shown in the Table 5 below and in FIG. 5. Table 5 provides data for IM delivery (mice nos. Z18-11 and Z18-12) and for systemic delivery

TABLE 5
CAPTN3 RT-PCR:

| | | | Dose of | | | | | | | |
|---|---|---|---|---|---|---|---|---|---|---|
| | | | Treatment | | | | | | | |
| | | | (DRAPs | CT Value | | | | | | |
| Mice No. | Tissue | Genotype | per Mice) | CAPN3 | | mGAPDH | | ΔCT | ΔΔCT | 2^{(-ΔΔCT)} |
| Z18-14* | TA | WT | 0 | 22.437 | 22.456 | 15.234 | 15.274 | 7.193 | 0.000 | 1.0003 |
| Z18-19 | TA | CAPN3 | 0 | 35.259 | 32.705 | 15.159 | 15.176 | 18.814 | 11.621 | 0.0003 |
| Z18-11 | TA | KO | 1E11 | 24.338 | 24.217 | 15.800 | 15.835 | 8.460 | 1.267 | 0.4155 |
| Z18-12 | TA | | | 21.030 | 21.104 | 14.906 | 15.058 | 6.085 | −1.108 | 2.1548 |
| Z18-13 | TA | | 3E12 | 32.376 | 32.430 | 15.236 | 15.203 | 17.183 | 9.990 | 0.0010 |
| Z18-15 | TA | | | 27.407 | 27.443 | 14.510 | 14.520 | 12.910 | 5.717 | 0.0190 |
| Z18-16 | TA | | | 28.609 | 28.333 | 15.229 | 15.259 | 13.227 | 6.034 | 0.0153 |
| Z18-17 | TA | | | 28.675 | 28.670 | 14.997 | 15.005 | 13.671 | 6.478 | 0.0112 |
| Z18-18 | TA | | | 27.869 | 28.128 | 14.522 | 14.544 | 13.466 | 6.273 | 0.0129 |
| Z18-20 | Gastroc | | 6E12 | 22.271 | 22.439 | 15.939 | 15.974 | 6.398 | −0.795 | 1.7347 |
| | Heart | | | 21.996 | 22.051 | 15.267 | 15.315 | 6.732 | −0.461 | 1.3762 |
| | Quadriceps | | | 21.008 | 21.202 | 15.203 | 15.407 | 5.800 | −1.393 | 2.6262 |
| | TA | | | 23.806 | 24.173 | 16.169 | 16.385 | 7.713 | 0.520 | 0.6975 |
| | Triceps | | | 24.083 | 24.361 | 15.978 | 16.097 | 8.185 | 0.992 | 0.5027 |
| Z18-21 | Gastroc | | | 25.330 | 25.221 | 15.461 | 15.462 | 9.814 | 2.621 | 0.1625 |
| | Heart | | | 25.024 | 24.819 | 15.032 | 15.097 | 9.857 | 2.664 | 0.1577 |
| | Quadriceps | | | 26.278 | 26.108 | 15.285 | 15.370 | 10.866 | 3.673 | 0.0784 |
| | TA | | | 26.649 | 26.697 | 15.017 | 15.010 | 11.659 | 4.466 | 0.0452 |
| | Triceps | | | 27.040 | 27.134 | 15.321 | 15.343 | 11.755 | 4.562 | 0.0423 |
| Z18-23 | Gastroc | | | 24.150 | 24.144 | 16.225 | 16.117 | 7.976 | 0.783 | 0.5812 |
| | Heart | | | 22.799 | 22.495 | 14.593 | 14.502 | 8.099 | 0.906 | 0.5335 |
| | Quadriceps | | | 24.248 | 24.076 | 16.511 | 16.504 | 7.655 | 0.462 | 0.7262 |
| | TA | | | 25.554 | 25.338 | 16.124 | 16.054 | 9.357 | 2.164 | 0.2231 |
| | Triceps | | | 24.396 | 24.383 | 15.363 | 15.277 | 9.070 | 1.877 | 0.2723 |
| Z18-24 | Gastroc | | | 24.444 | 24.165 | 18.083 | 18.036 | 6.245 | −0.948 | 1.9297 |
| | Heart | | | 22.769 | 22.425 | 15.100 | 15.077 | 7.508 | 0.315 | 0.8037 |
| | Quadriceps | | | 22.754 | 22.521 | 15.637 | 15.672 | 6.983 | −0.210 | 1.1568 |
| | TA | | | 23.491 | 23.555 | 15.979 | 15.974 | 7.547 | 0.354 | 0.7826 |
| | Triceps | | | 24.554 | 24.403 | 16.370 | 16.329 | 8.128 | 0.935 | 0.5229 |
| Z18-22 | Gastroc | | 0 | 31.878 | 31.962 | 15.433 | 15.527 | 16.440 | 9.247 | 0.0016 |
| | Heart | | | 31.407 | 32.964 | 16.006 | 15.972 | 16.197 | 9.004 | 0.0019 |
| | Quadriceps | | | 32.332 | 33.464 | 16.528 | 16.468 | 16.400 | 9.207 | 0.0017 |
| | TA | | | 35.584 | 33.917 | 16.451 | 16.372 | 18.339 | 11.146 | 0.0004 |
| | Triceps | | | 33.615 | 32.786 | 15.742 | 15.628 | 17.516 | 10.323 | 0.0008 |
| Human Tissue | Muscle | | | 23.547 | 23.539 | 37.743 | 38.262 | | | |
| pAAV.tMCK. | 1 pg/uL | | | 16.328 | 16.413 | UD | 38.302 | | | |
| hCAPN3 | | | | | | | | | | |

*Calibrator

Overall, the CAPN3 mRNA expression in the CAPN3 KO muscle following systemic delivery had animal-and tissue-specific variability and lower relative expression as compared to the IM delivery at 1E11 vg (<1% of IM delivery); this was especially true for the 3E12 low dose cohort. Accordingly, the full-length 94 kDa protein was below the limit of detection by Western blot. However, robust gene expression and prominent amounts of full-length Calpain 3 protein were exhibited following systemic injection of 6E12 vg systemic dosage in the high dose cohort.

### Example 10

### Assessment of Systemic AAVrh74.tMCK.hCAPN3 Gene Delivery

Gene transfer efficiency was assessed by qPCR, calculating vector genome copies within CAPN3 KO mouse tissue samples following systemic delivery of AAVrh74.tMCK.hCAPN3 at 6E12 vg. The vector genome load of the lower and upper extremity skeletal muscles (quad, TA, gastroc, triceps), heart and liver was determined. Genomic DNA was isolated from frozen tissue samples. The qPCR assay was performed on an ABI 7500 (Applied Biosystems) using the following primer set: “5′-CGGAGAGCAACTGCATAAG-3′ (Forward; SEQ ID NO: 8);” 5′-GGCTGATGATGGCTGAATAG-3′ (Reverse; SEQ ID NO: 9). The primer pair exclusively amplifies a product from the 5′ region of the hCAPN3 ORF, and region downstream unique to the expression vector, including portions of an intronic element. The final results are reported as mean copy number of AAVrh74 vector per microgram of genomic DNA.

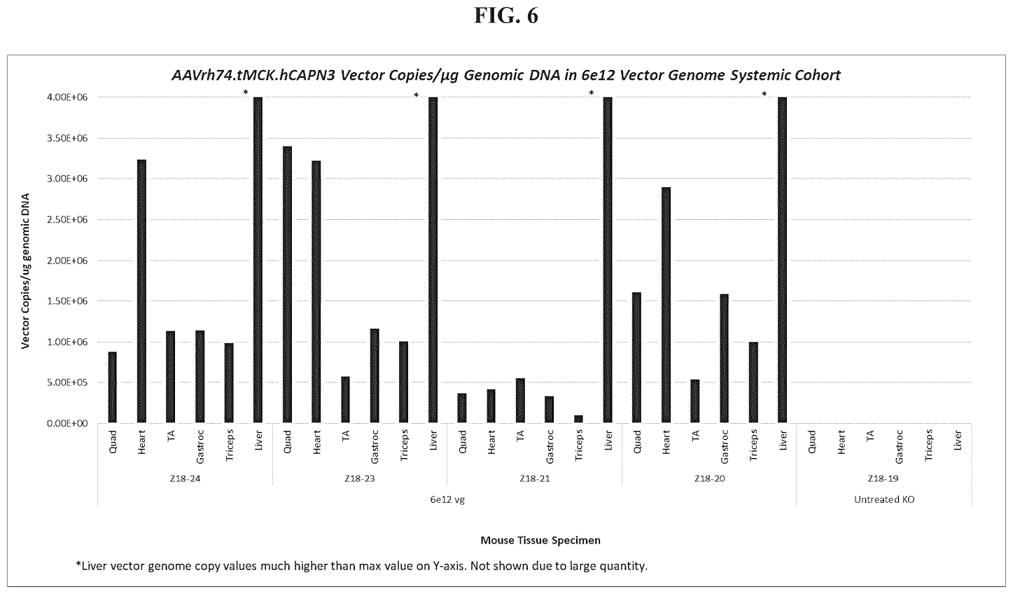

As shown in FIG. 6, the highest vector genome copy number was present in the liver following systemic vector delivery. Vector genome distribution was variable between the muscle groups. Overall the values were higher in the quadriceps and heart tissue compared to other muscles. Experimental variability was also noted; as the case with Mouse no. Z18-21 which showed relatively lower copy numbers in all muscle groups compared to other 3 mice.

Improvement in both functional and histological features were observed in the 3E12 vg systemically treated CAPN3 KO mice, however, only low levels of muscle Calpain 3 expression were detected in total RNA isolates by RT-qPCR and the full-length 94 kDa protein was undetectable by Western blot for the particular muscle tissue (See FIG. 3A). However, robust gene expression and prominent amounts of full-length Calpain 3 protein were exhibited following the 6E12 vg systemic dosage (see FIG. 3B). The data demonstrates that Calpain 3 gene expression returned to normalized levels as compared to WT mice after 4 weeks post-gene transfer of the AAVrh74.tMCK.hCAPN3 particles. Mouse GAPDH was used as a reference gene and WT C57BL/6 to calibrate the RT-qPCR data.

### Histopathology

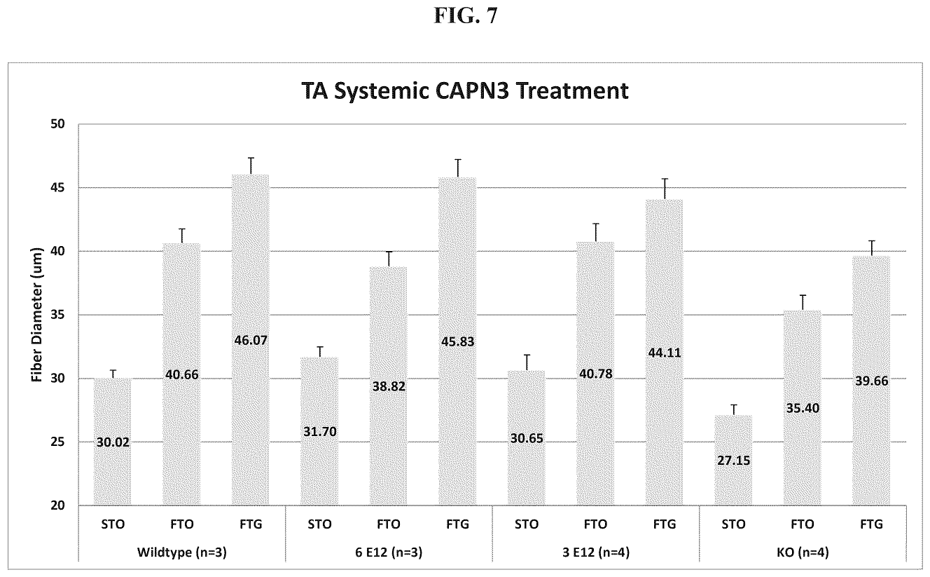

As discussed above, an efficacy trend at 4 weeks post-injection was observed. A significant increase in fiber size was observed in the TA muscle from CAPN3 KO mice following systemic delivery of AAVrh.74.tMCK.hCAPN3 at 4 week-post injection in both cohorts (3E12 and 6E12). As shown in FIG. 7, total fiber diameter was significantly increased in both of the treated cohorts compared to untreated KO counterparts (p<0.00001). Treatment resulted in normalization of fiber size and there was no dose-related difference between the treatment cohorts (p=0.78058). Table 6 provides the muscle fiber sizes in wild type and CAPN3 KO mice following systemic AAV.hCAPN3 gene therapy at 3E12 and 6E12 vg.

TABLE 6

| | WT (n = 3) | | 6 E12 CAPN3 (n = 3) | | 3E12 CAPN3 (n = 4) | | KO (n = 4) | |
|---|---|---|---|---|---|---|---|---|
| | number | diameter (μm) | number | diameter (μm) | number | diameter (μm) | number | diameter (μm) |
| STO | 532 | 30.0 ± 0.6 | 441 | 31.7 ± 0.8 | 464 | 30.7 ± 1.2 | 858 | 27.2 ± 0.8 |
| FTO | 278 | 40.7 ± 1.1 | 345 | 38.8 ± 1.1 | 447 | 40.8 ± 1.4 | 364 | 35.4 ± 1.1 |
| FTG | 275 | 46.1 ± 1.3 | 226 | 45.8 ± 1.4 | 403 | 44.1 ± 1.6 | 455 | 39.7 ± 1.2 |
| All fiber | 1085 | 38.9 ± 1.8 | 1012 | 38.8 ± 2.0 | 1314 | 38.5 ± 2.4 | 1677 | 34.1 ± 1.8 |

There was no histopathological evidence of cardiac toxicity following systemic injection of AAVrh7.4.tMCK.hCAPN3 vector at 4 weeks in either cohort. There were variable amounts of virus found in the heart tissue, however no protein bands were detected in the heart tissue by Western blot in either cohort.

### Functionality Study: Run-to-Exhaustion Test

Mice were accustomed to the treadmill (Columbus Instruments) with a 15-minute run once per day at 10 m/min for 3 days prior to data acquisition for Run-to-Exhaustion test. The protocol used required having mice on a treadmill that is at a 15-degree incline. The treadmill was turned on at a speed at 1 m/min and the speed was increased by 1 m every minute until the mouse got exhausted. Exhaustion was determined when the mouse sits on the rest pad for at least 15 seconds. The time, speed and distance to exhaustion were recorded.

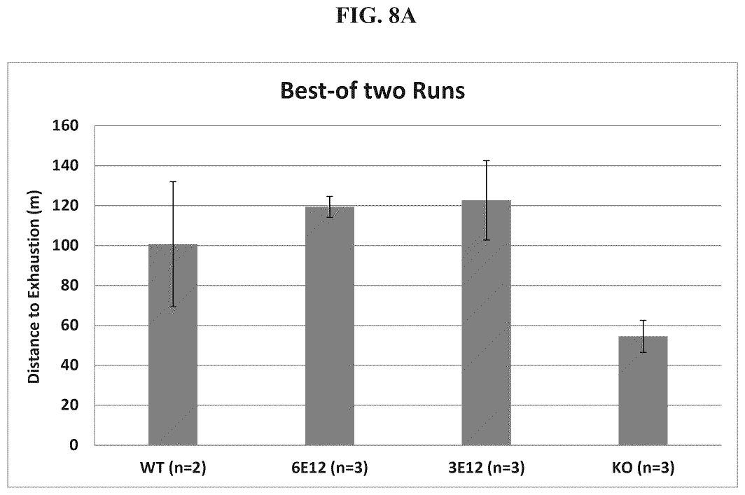

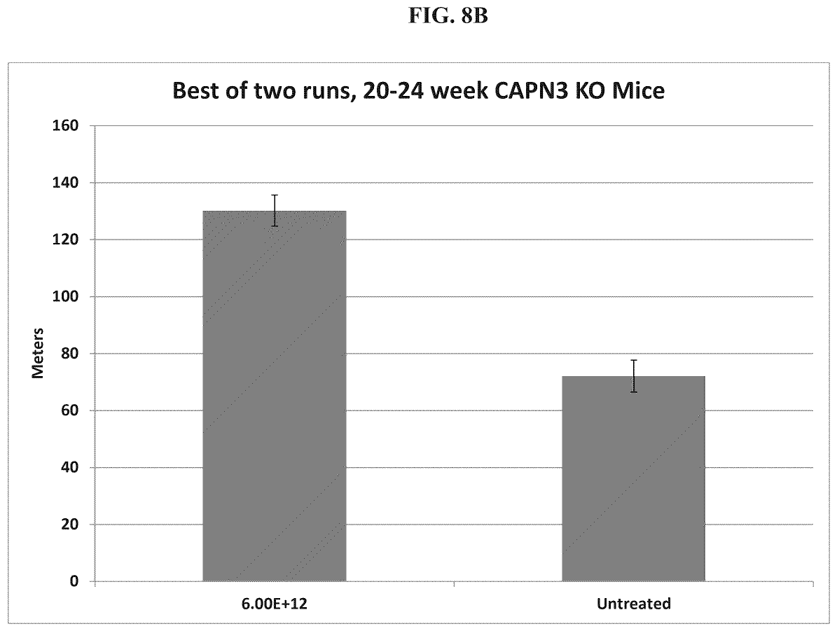

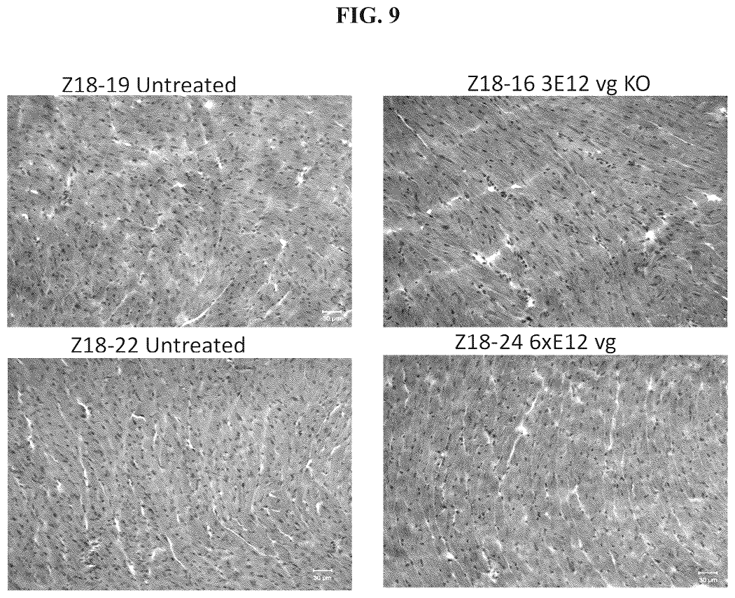

FIG. 8A provides data for the run-to-exhaustion test for the low dose cohort, which received 3E12 vg of AAVrh7.4.tMCK.hCAPN3, and the high dose cohort 2, which received 6E12 vg of AAVrh7.4.tMCK.hCAPN3 as assessed 4 weeks after systemic administration. Treated CAPN3 KO mice in both cohorts performed better on the Run-to-Exhaustion test compared to untreated counterparts. There was no apparent dose-related difference in the Run-to-Exhaustion test performance or statistical difference in muscle fiber diameter between the low and high dose cohorts.

Mice from the high dose cohort 2 (n=16) were further analyzed 20-24 weeks after administration of 6E12 vg of AAVrh7.4.tMCK.hCAPN3. As shown in FIG. 8B, the treated CAPN3 KO mice continued to perform better on the Run-to-Exhaustion test compared to untreated counterparts (p<0.00001).

### Example 11

### Assessment of Cardiac Toxicity Following Systemic Injection of AAVrh7.4.tMCK.hCAPN3 Vector

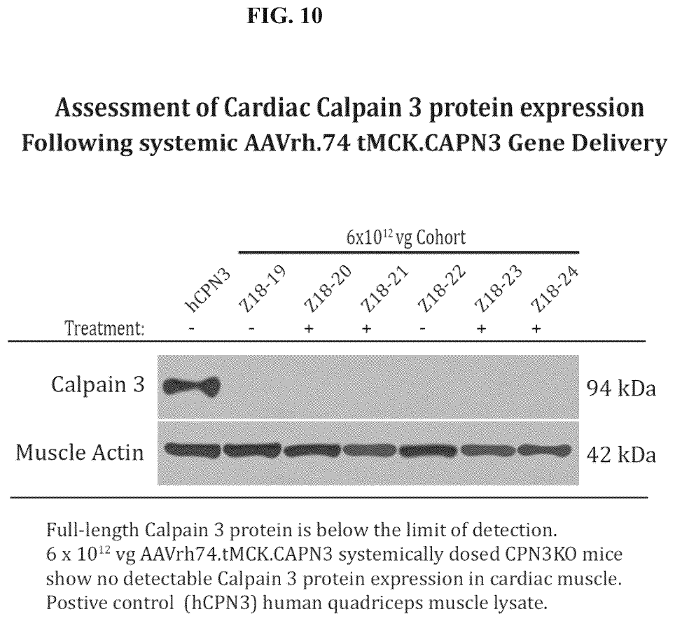

After the mice of the cohorts were euthanized at 4 weeks post injection, serum and organ samples were collected. The low dose cohort 1 CAPN3KO cohort (n=5) received 3E12 vg in 300 μl Ringer's lactate of AAVrh.74.tMCK.hCAPN3 vector via tail vein injection. The high dose cohort 2CAPN3-KO mice received 6E12 vg AAVrh7.4.tMCK.hCAPN3 vector via tail vein, and both cohorts were euthanized 4 weeks post-injection. Two sections through the apex of the heart, superficial and deep regions of ventricles were examined. No inflammation, necrosis or regeneration was found in the tissue sections indicating no toxic effects were observed on the heart muscle from the systemic delivery of AAVrh7.4.tMCK.hCAPN3 vector at two different doses at 4 weeks post-injection. Mice nos. Z18-19 and Z18-22 (Ringer's lactate-injected/untreated) served as control KO animal. FIG. 9 provides H&E stained fresh frozen sections from the heart. Muscle fiber necrosis, regeneration or inflammation was not seen. Even though there were variable amounts of viruses present in the heart tissue, no protein bands were detected by Western blot in either cohort. FIG. 10 provides the Western blot analysis which shows the full-length Calpain 3 protein is below the limit of detection in the heart tissues after the transduction.

### Example 12

### In Vivo Physiological Analysis

Physiological assessment is carried out after IM or systemic administration of the AAVrh7.4.tMCK.hCAPN3 vector. During the in vivo physiological assessments, the mice are anesthetized with inhaled isoflurane. Once the animal is anesthetized, the hair from the back and the hind limb is removed as needed with clippers. If hair removal with clippers is insufficient, a thin layer of hair-removal cream is applied. During in vivo physiological force measurements, torque from the hind limb is measured with a non-invasive force foot plate connected to force detecting motor (Aurora Scientific, Canada) following supramaximal stimulations of the sciatic nerve. The hind limb to be measured is attached to the foot plate with adhesive tape. The limb is held rigid in a blunt clamp. Either the tibial or peroneal component of the sciatic nerve is stimulated with two sterile disposable 28 gauge monopolar electrodes inserted subcutaneously near the nerve. Mouse temperature is maintained by conductive thermoregulated heating plate (set at 37° C.) or radiant heat source and monitored by infrared temperature probe.

While the present disclosure provides specific embodiments, it is understood that variations and modifications will occur to those skilled in the art. Accordingly, only such limitations as appear in the claims should be placed on the invention.

All documents referred to in this application are hereby incorporated by reference in their entirety.

We claim:

1. A recombinant adeno-associated virus (rAAV) comprising a polynucleotide comprising a sequence which is at least 95% identical to nucleotides 1 to 3977 of SEQ ID NO: 1.

2. The rAAV of claim 1, wherein the polynucleotide comprises a sequence which is at least 96%, 97%, 98%, or 99% identical to nucleotides 1 to 3977 of SEQ ID NO: 1.

3. The rAAV of claim 1, wherein the rAAV comprises a capsid protein selected from AAV-1, AAV-2, AAV-3, AAV-4, AAV-5, AAV-6, AAV-7, AAV-8, AAV-9, AAV-10, AAV-11, AAV-12, AAV-13, AAV rh.74, AAV rh.10 capsid protein, or a variant of each thereof.

4. A composition comprising the rAAV of claim 1 and a pharmaceutically acceptable carrier.

5. A composition for treating limb girdle muscular dystrophy 2A comprising a therapeutically effective amount of the rAAV of claim 1 and a pharmaceutically acceptable carrier.

6. The composition of claim 5, wherein the composition is formulated for administration by intramuscular injection or intravenous injection.

7. A method of treating limb girdle muscular dystrophy 2A in a subject comprising administering to the subject a therapeutically effective amount of a recombinant adeno-associated virus (rAAV) comprising a polynucleotide comprising a sequence which is at least 95% identical to nucleotides 1 to 3977 of SEQ ID NO: 1.

8. The method of claim 7, wherein the polynucleotide comprises a sequence which is at least 96%, 97%, 98%, or 99% identical to nucleotides 1 to 3977 of SEQ ID NO: 1.

9. The method of claim 7, wherein the rAAV comprises one or more capsid proteins selected from an AAV serotype selected from AAV-1, AAV-2, AAV-3, AAV-4, AAV-5, AAV-6, AAV-7, AAV-8, AAV-9, AAV-10, AAV-11, AAV-12, AAV-13, AAV rh.74, AAV rh.10 capsid protein, or a variant of each thereof.

10. The method of claim 7, wherein the treatment results in one or more of:
(a) an increased muscle fiber diameter,
(b) a decreased number of small lobulated muscle fibers,
(c) a decreased number of fibers with internal nuclei,
(d) a decreased endomysial connective tissue content,
(e) correction of muscle atrophy, and
(f) an increased muscle force generation.

11. The method of claim 10, wherein the muscle fibers comprise one or more of slow twitch oxidative (STO) muscle fiber, fast twitch oxidative (FTO) muscle fiber, and fast twitch glycolytic (FTG) fiber.

12. The method of claim 7, wherein the treatment results in one or more of:
(a) at least a 5%, 10%, 15%, 20%, 25%, 30%, or 35%, or 40% decrease of total muscle fiber number per mm² by 4 weeks after administration;
(b) at least a 5%, 10%, 15%, 20%, or 25% increase of muscle fiber diameter by 4 weeks after administration;
(c) at least a 5%, 10%, 15%, 20%, 25%, 30%, 35%, or 42% decrease of STO muscle fiber number per mm² by 4 weeks after administration;
(d) at least a 5%, 10%, 15%, 20%, or 25% increase of STO muscle fiber diameter by 4 weeks after administration;
(e) at least a 5%, 10%, 15%, or 20% decrease of FTO muscle fiber number per mm² by 4 weeks after administration;
(f) at least a 5%, 10%, 15%, or 20% increase of FTO muscle fiber diameter by 4 weeks after administration;
(g) at least a 5%, 10%, 15%, 20%, 25%, 30%, or 35% decrease of FTG muscle fiber number per mm² by 4 weeks after administration; and
(h) at least a 5%, 10%, 15%, 20%, or 25% increase of FTG muscle fiber diameter by 4 weeks after administration.

13. The method of claim 7, wherein the administration comprises intramuscular injection or intravenous injection.

14. The method of claim 10, wherein the heart muscle of the subject shows minimum or low calpain 3 protein expressed from the rAAV.

15. A recombinant adeno-associated virus rh.74 (rAAVrh.74) comprising a polynucleotide comprising a first AAV inverted terminal repeat (ITR), a promoter comprising the nucleotide sequence of SEQ ID NO: 3, a nucleotide sequence comprising the nucleotide sequence of SEQ ID NO: 2 encoding a protein with calpain 3 (CAPN3) activity and a second AAV ITR.

16. A composition comprising the rAAVrh.74 of claim 15 and a pharmaceutically acceptable carrier.

17. A method of treating limb girdle muscular dystrophy 2A in a subject comprising administering to the subject a therapeutically effective amount of the rAAVrh.74 of claim 15.

18. The method of claim 17, wherein the treatment results in one or more of:
(a) an increased muscle fiber diameter,
(b) a decreased number of small lobulated muscle fibers,
(c) a decreased number of fibers with internal nuclei,
(d) a decreased endomysial connective tissue content,
(e) correction of muscle atrophy, and
(f) an increased muscle force generation.

19. The method of claim 17, wherein the treatment results in one or more of:
(a) at least a 5%, 10%, 15%, 20%, 25%, 30%, or 35%, or 40% decrease of total muscle fiber number per mm² by 4 weeks after administration;
(b) at least a 5%, 10%, 15%, 20%, or 25% increase of muscle fiber diameter by 4 weeks after administration;
(c) at least a 5%, 10%, 15%, 20%, 25%, 30%, 35%, or 42% decrease of STO muscle fiber number per mm² by 4 weeks after administration;
(d) at least a 5%, 10%, 15%, 20%, or 25% increase of STO muscle fiber diameter by 4 weeks after administration;
(e) at least a 5%, 10%, 15%, or 20% decrease of FTO muscle fiber number per mm² by 4 weeks after administration;
(f) at least a 5%, 10%, 15%, or 20% increase of FTO muscle fiber diameter by 4 weeks after administration;
(g) at least a 5%, 10%, 15%, 20%, 25%, 30%, or 35% decrease of FTG muscle fiber number per mm² by 4 weeks after administration; and
(h) at least a 5%, 10%, 15%, 20%, or 25% increase of FTG muscle fiber diameter by 4 weeks after administration.

20. The method of claim 17, wherein the administration comprises intramuscular injection or intravenous injection.


SEQUENCE LISTING (9 sequences)

SEQ ID NO: 1 - DNA, 7629 bp, synthetic construct
  misc_feature 1..7629: note = Synthetic polynucleotide
  misc_feature 1..7629: note = AAVrh.74.tMCK.CAPN3 recombinant genome
  misc_feature 1..128: note = AAV2 ITR
  misc_feature 165..884: note = tMCK promoter
  misc_feature 937..1069: note = chimeric intron
  misc_feature 1101..1106: note = Kozak Sequence
  misc_feature 1107..3572: note = CAPN3 polynucleotide
  misc_feature 3581..3780: note = poly A signal
  misc_feature 3850..3977: note = second AAV2 ITR
  source 1..7629: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttaatacgactcactatagggccgcaccggtaagcttccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatcctccctggggacagcccctcctggctagtcacaccctgtaggctcctctatataacccaggggcacaggggctgcccccgggtcacggtggatatccctgcaggtaccacgcgctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacggagagcaactgcataagggctagcctcgaggccaccatgccgaccgtcattagcgcatctgtggctccaaggacagcggctgagccccggtccccagggccagttcctcacccggcccagagcaaggccactgaggctgggggtggaaacccaagtggcatctattcagccatcatcagccgcaattttcctattatcggagtgaaagagaagacattcgagcaacttcacaagaaatgtctagaaaagaaagttctttatgtggaccctgagttcccaccggatgagacctctctcttttatagccagaagttccccatccagttcgtctggaagagacctccggaaatttgcgagaatccccgatttatcattgatggagccaacagaactgacatctgtcaaggagagctaggggactgctggtttctcgcagccattgcctgcctgaccctgaaccagcaccttcttttccgagtcataccccatgatcaaagtttcatcgaaaactacgcagggatcttccacttccagttctggcgctatggagagtgggtggacgtggttatagatgactgcctgccaacgtacaacaatcaactggttttcaccaagtccaaccaccgcaatgagttctggagtgctctgctggagaaggcttatgctaagctccatggttcctacgaagctctgaaaggtgggaacaccacagaggccatggaggacttcacaggaggggtggcagagttttttgagatcagggatgctcctagtgacatgtacaagatcatgaagaaagccatcgagagaggctccctcatgggctgctccattgatgatggcacgaacatgacctatggaacctctccttctggtctgaacatgggggagttgattgcacggatggtaaggaatatggataactcactgctccaggactcagacctcgaccccagaggctcagatgaaagaccgacccggacaatcattccggttcagtatgagacaagaatggcctgcgggctggtcagaggtcacgcctactctgtcacggggctggatgaggtcccgttcaaaggtgagaaagtgaagctggtgcggctgcggaatccgtggggccaggtggagtggaacggttcttggagtgatagatggaaggactggagctttgtggacaaagatgagaaggcccgtctgcagcaccaggtcactgaggatggagagttctggatgtcctatgaggatttcatctaccatttcacaaagttggagatctgcaacctcacggccgatgctctgcagtctgacaagcttcagacctggacagtgtctgtgaacgagggccgctgggtacggggttgctctgccggaggctgccgcaacttcccagatactttctggaccaaccctcagtaccgtctgaagctcctggaggaggacgatgaccctgatgactcggaggtgatttgcagcttcctggtggccctgatgcagaagaaccggcggaaggaccggaagctaggggccagtctcttcaccattggcttcgccatctacgaggttcccaaagagatgcacgggaacaagcagcacctgcagaaggacttcttcctgtacaacgcctccaaggccaggagcaaaacctacatcaacatgcgggaggtgtcccagcgcttccgcctgcctcccagcgagtacgtcatcgtgccctccacctacgagccccaccaggagggggaattcatcctccgggtcttctctgaaaagaggaacctctctgaggaagttgaaaataccatctccgtggatcggccagtgaaaaagaaaaaaaccaagcccatcatcttcgtttcggacagagcaaacagcaacaaggagctgggtgtggaccaggagtcagaggagggcaaaggcaaaacaagccctgataagcaaaagcagtccccacagccacagcctggcagctctgatcaggaaagtgaggaacagcaacaattccggaacattttcaagcagatagcaggagatgacatggagatctgtgcagatgagctcaagaaggtccttaacacagtcgtgaacaaacacaaggacctgaagacacacgggttcacactggagtcctgccgtagcatgattgcgctcatggatacagatggctctggaaagctcaacctgcaggagttccaccacctctggaacaagattaaggcctggcagaaaattttcaaacactatgacacagaccagtccggcaccatcaacagctacgagatgcgaaatgcagtcaacgacgcaggattccacctcaacaaccagctctatgacatcattaccatgcggtacgcagacaaacacatgaacatcgactttgacagtttcatctgctgcttcgttaggctggagggcatgttcagagcttttcatgcatttgacaaggatggagatggtatcatcaagctcaacgttctggagtggctgcagctcaccatgtatgcctgagcggccgcggggatccagacatgataagatacattgatgagtttggacaaaccacaactagaatgcagtgaaaaaaatgctttatttgtaaaatttgtgatgctattgctttatttgtaaccattataagctgcaataaacaagttaacaacaacaattgcattcattttatgtttcaggttcagggggaggtgtgggaggttttttcggatcctctagagtcgaccagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaacttccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaatcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagct

SEQ ID NO: 2 - DNA, 2466 bp, synthetic construct
  misc_feature 1..2466: note = Synthetic polynucleotide
  misc_feature 1..2466: note = CAPN3 polynucleotide
  source 1..2466: mol_type = other DNA
atgccgaccgtcattagcgcatctgtggctccaaggacagcggctgagccccggtccccagggccagttcctcacccggcccagagcaaggccactgaggctgggggtggaaacccaagtggcatctattcagccatcatcagccgcaattttcctattatcggagtgaaagagaagacattcgagcaacttcacaagaaatgtctagaaaagaaagttctttatgtggaccctgagttcccaccggatgagacctctctcttttatagccagaagttccccatccagttcgtctggaagagacctccggaaatttgcgagaatccccgatttatcattgatggagccaacagaactgacatctgtcaaggagagctaggggactgctggtttctcgcagccattgcctgcctgaccctgaaccagcaccttcttttccgagtcataccccatgatcaaagtttcatcgaaaactacgcagggatcttccacttccagttctggcgctatggagagtgggtggacgtggttatagatgactgcctgccaacgtacaacaatcaactggttttcaccaagtccaaccaccgcaatgagttctggagtgctctgctggagaaggcttatgctaagctccatggttcctacgaagctctgaaaggtgggaacaccacagaggccatggaggacttcacaggaggggtggcagagttttttgagatcagggatgctcctagtgacatgtacaagatcatgaagaaagccatcgagagaggctccctcatgggctgctccattgatgatggcacgaacatgacctatggaacctctccttctggtctgaacatgggggagttgattgcacggatggtaaggaatatggataactcactgctccaggactcagacctcgaccccagaggctcagatgaaagaccgacccggacaatcattccggttcagtatgagacaagaatggcctgcgggctggtcagaggtcacgcctactctgtcacggggctggatgaggtcccgttcaaaggtgagaaagtgaagctggtgcggctgcggaatccgtggggccaggtggagtggaacggttcttggagtgatagatggaaggactggagctttgtggacaaagatgagaaggcccgtctgcagcaccaggtcactgaggatggagagttctggatgtcctatgaggatttcatctaccatttcacaaagttggagatctgcaacctcacggccgatgctctgcagtctgacaagcttcagacctggacagtgtctgtgaacgagggccgctgggtacggggttgctctgccggaggctgccgcaacttcccagatactttctggaccaaccctcagtaccgtctgaagctcctggaggaggacgatgaccctgatgactcggaggtgatttgcagcttcctggtggccctgatgcagaagaaccggcggaaggaccggaagctaggggccagtctcttcaccattggcttcgccatctacgaggttcccaaagagatgcacgggaacaagcagcacctgcagaaggacttcttcctgtacaacgcctccaaggccaggagcaaaacctacatcaacatgcgggaggtgtcccagcgcttccgcctgcctcccagcgagtacgtcatcgtgccctccacctacgagccccaccaggagggggaattcatcctccgggtcttctctgaaaagaggaacctctctgaggaagttgaaaataccatctccgtggatcggccagtgaaaaagaaaaaaaccaagcccatcatcttcgtttcggacagagcaaacagcaacaaggagctgggtgtggaccaggagtcagaggagggcaaaggcaaaacaagccctgataagcaaaagcagtccccacagccacagcctggcagctctgatcaggaaagtgaggaacagcaacaattccggaacattttcaagcagatagcaggagatgacatggagatctgtgcagatgagctcaagaaggtccttaacacagtcgtgaacaaacacaaggacctgaagacacacgggttcacactggagtcctgccgtagcatgattgcgctcatggatacagatggctctggaaagctcaacctgcaggagttccaccacctctggaacaagattaaggcctggcagaaaattttcaaacactatgacacagaccagtccggcaccatcaacagctacgagatgcgaaatgcagtcaacgacgcaggattccacctcaacaaccagctctatgacatcattaccatgcggtacgcagacaaacacatgaacatcgactttgacagtttcatctgctgcttcgttaggctggagggcatgttcagagcttttcatgcatttgacaaggatggagatggtatcatcaagctcaacgttctggagtggctgcagctcaccatgtatgcctga

SEQ ID NO: 3 - DNA, 720 bp, synthetic construct
  misc_feature 1..720: note = Synthetic polynucleotide
  misc_feature 1..720: note = tMCK promoter
  source 1..720: mol_type = other DNA
ccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatcctccctggggacagcccctcctggctagtcacaccctgtaggctcctctatataacccaggggcacaggggctgcccccgggtcac

SEQ ID NO: 4 - DNA, 22 bp, synthetic construct
  misc_feature 1..22: note = Synthetic polynucleotide
  misc_feature 1..22: note = MCK forward primer
  source 1..22: mol_type = other DNA
cccgagatgcctggttataatt

SEQ ID NO: 5 - DNA, 19 bp, synthetic construct
  misc_feature 1..19: note = Synthetic polynucleotide
  misc_feature 1..19: note = MCK reverse primer
  source 1..19: mol_type = other DNA
gctcaggcagcaggtgttg

SEQ ID NO: 6 - DNA, 22 bp, synthetic construct
  misc_feature 1..22: note = Synthetic polynucleotide
  misc_feature 1..22: note = MCK probe
  source 1..22: mol_type = other DNA
ccagacatgtggctgctccccc

SEQ ID NO: 7 - protein, 821 aa, Homo sapiens
MPTVISASVAPRTAAEPRSPGPVPHPAQSKATEAGGGNPSGIYSAIISRNFPIIGVKEKTFEQLHKKCLEKKVLYVDPEFPPDETSLFYSQKFPIQFVWKRPPEICENPRFIIDGANRTDICQGELGDCWFLAAIACLTLNQHLLFRVIPHDQSFIENYAGIFHFQFWRYGEWVDVVIDDCLPTYNNQLVFTKSNHRNEFWSALLEKAYAKLHGSYEALKGGNTTEAMEDFTGGVAEFFEIRDAPSDMYKIMKKAIERGSLMGCSIDDGTNMTYGTSPSGLNMGELIARMVRNMDNSLLQDSDLDPRGSDERPTRTIIPVQYETRMACGLVRGHAYSVTGLDEVPFKGEKVKLVRLRNPWGQVEWNGSWSDRWKDWSFVDKDEKARLQHQVTEDGEFWMSYEDFIYHFTKLEICNLTADALQSDKLQTWTVSVNEGRWVRGCSAGGCRNFPDTFWTNPQYRLKLLEEDDDPDDSEVICSFLVALMQKNRRKDRKLGASLFTIGFAIYEVPKEMHGNKQHLQKDFFLYNASKARSKTYINMREVSQRFRLPPSEYVIVPSTYEPHQEGEFILRVFSEKRNLSEEVENTISVDRPVKKKKTKPIIFVSDRANSNKELGVDQESEEGKGKTSPDKQKQSPQPQPGSSDQESEEQQQFRNIFKQIAGDDMEICADELKKVLNTVVNKHKDLKTHGFTLESCRSMIALMDTDGSGKLNLQEFHHLWNKIKAWQKIFKHYDTDQSGTINSYEMRNAVNDAGFHLNNQLYDIITMRYADKHMNIDFDSFICCFVRLEGMFRAFHAFDKDGDGIIKLNVLEWLQLTMYA

SEQ ID NO: 8 - DNA, 19 bp, synthetic construct
  misc_feature 1..19: note = Synthetic Primer
  source 1..19: mol_type = other DNA
cggagagcaactgcataag

SEQ ID NO: 9 - DNA, 20 bp, synthetic construct
  misc_feature 1..20: note = Synthetic Primer
  source 1..20: mol_type = other DNA
ggctgatgatggctgaatag